(19) United States
(12) Patent Application Publication
(10) Pub. No.: US 2009/0214599 A1
(43) Pub. Date: Aug. 27, 2009

(54) PROTON PUMP INHIBITOR FORMULATIONS, AND METHODS OF PREPARING AND USING SUCH FORMULATIONS

(75) Inventors: John DEVANE, Athlone (IE); David YOUNG, Ellicot City, MD (US)

Correspondence Address: GREENBLUM & BERNSTEIN, P.L.C., 1950 ROLAND CLARKE PLACE, RESTON, VA, 20191, US

(73) Assignee: AGI THERAPEUTICS PLC, Dublin (IE)

(21) Appl. No.: 12/035,147

(22) Filed: Feb. 21, 2008

(51) Int. Cl. A61K 9/00; A61K 47/30; A61K 31/4439; A61P 1/04

(52) U.S. Cl. 424/400; 514/772.3; 514/338

(57) ABSTRACT

Pharmaceutical formulation comprising at least one proton pump inhibitor structured and arranged to provide an initial pH-dependent delayed release, and a pH-dependent extended release of the at least one proton pump inhibitor.

### BACKGROUND OF THE INVENTION

1. Field of the Invention

The present invention is directed to proton pump inhibitors (PPIs), to formulations containing proton pump inhibitors, to formulations containing proton pump inhibitors that are constructed and arranged to provide unique PPI release rates, and particularly to formulations designed to treat gastric acid related conditions, especially to counteract nocturnal acid breakthrough. The formulations according to the present invention particularly comprise proton pump inhibitor formulations that have a pH-dependent protective layer, and exhibit a pH-dependent extended release. The present invention is also directed to methods of using proton pump inhibitors, such as in the treatment of gastric acid related conditions, including methods wherein the proton pump inhibitor is administered in a formulation that provides pH-dependent extended release of the proton pump inhibitor. The formulations of the present invention can be used to treat nocturnal acid breakthrough, either alone or in combination with other formulations. The present invention is also directed to methods of preparing such formulations.

2. Discussion of Background Information

Omeprazole is a proton pump inhibitor (PPI) and is currently marketed as PRILOSEC® (^{PR}LOSEC®; omeprazole delayed-release capsules or tablets) which is indicated for the treatment of heartburn and other symptoms associated with gastro-esophageal reflux disease (GERD) (20 mg/day) including the short-term treatment of erosive esophagitis which has been diagnosed by endoscopy and to maintain healing of erosive esophagitis (20 mg/day) (PRILOSEC® Product Label (US). PDR 2004, 633-638).

PRILOSEC® is also indicated for the short-term treatment of active duodenal ulcer (20 mg/day) and active benign gastric ulcer (40 mg/day) and for the long-term treatment of pathological hypersecretory conditions (e.g., Zollinger-Ellison syndrome, multiple endocrine adenomas and systemic mastocytosis—60-360 mg/day) (PRILOSEC® Product Label (US). PDR 2004, 633-638). PRILOSEC® (20-40 mg/day), in combination with clarithromycin and amoxicillin, is also indicated for the treatment of patients with H. pylori infection and active duodenal ulcer to eradicate H. pylori. (PRILOSEC® Product Label (US). PDR 2004, 633-638.)

PRILOSEC® Delayed-Release Capsules and Tablets contain an enteric-coating formulation of omeprazole (because omeprazole is acid-labile), so that release of omeprazole begins only after the dosage form leaves the stomach. The enteric coating is formulated to provide an intact protective barrier at pH values <5.5 but the enteric polymer (Eudragit L) rapidly dissolves at pH values >5.5. This pH value coincides with the transition from the stomach contents to those in the upper small intestine. Thus, these dosage forms provide for a delayed release followed by a rapid release. The rate of degradation in acid conditions has been reported to be very rapid, i.e. about a 10 minutes half-life, in pH <4. However even in less acidic conditions up to neutral pH and beyond, omeprazole still degrades and in pH conditions known to occur in the intestines the half-life is still short. For an extended release formulation of a PPI, designed to release the PPI gradually within the intestines the in-situ intestinal pH will result in substantial degradation of the PPI. This pH-lability of omeprazole is shared with all the other marketed PPIs, which are also formulated as enteric-coated delayed rapid release tablets or capsules.

Absorption of omeprazole following ingestion of the marketed delayed release forms is rapid, with peak plasma levels of omeprazole occurring within 0.5 to 3.5 hours. Peak plasma concentrations of omeprazole and AUC are approximately proportional to doses up to 40 mg, but because of a saturable first-pass effect, a greater than linear response in peak plasma concentration and AUC occurs with doses greater than 40 mg. Absolute bioavailability (compared to intravenous administration) is about 30-40% at doses of 20-40 mg, due in large part to presystemic metabolism. In healthy subjects, the plasma half-life is 0.5 to 1 hour, and the total body clearance is 500-600 mL/min. Protein binding is approximately 95%. (PRILOSEC® Product Label (US). PDR 2004, 633-638.) In the light of the pH instability of PPIs in the pH conditions of the intestines, traditional modified release systems that release the PPI largely independent of pH, and typically, with first order or zero-order release rates will suffer from substantial degradation of the PPI.

GERD refers to the symptoms and/or tissue injury related to the reflux of gastric contents into the esophagus. Heartburn is the most common symptom and over time, there will be damage to the esophagus. A proportion (10%) will develop Barrett's esophagus, which increases the risk of cancer of the esophagus. GERD is one of the most common complaints encountered in general medical practice. It has been estimated that 44% of adults in the United States experience heartburn at least once a month (Ofman, J. J., “The economic and quality-of-life impact of symptomatic gastroesophageal reflux disease,” Am. J. Gastroenterol. 98(3 Suppl): S8-S14 (March 2003)). GERD has considerable adverse effects on work productivity (Dean B B, Crawley J A, Schmitt C M, Wong J, Ofman J J. The burden of illness of gastro-oesophageal reflux disease: impact on work productivity. Aliment Pharmacol Ther. May 15, 2003; 17(10):1309-17) and quality of life (Ofman, J. J., “The economic and quality-of-life impact of symptomatic gastroesophageal reflux disease,” Am. J. Gastroenterol. 98(3 Suppl): S8-S14 (March 2003)) in those affected. Symptom severity and night-time heartburn are significantly associated with productivity and quality of life, particularly when nocturnal heartburn interferes with sleep (Dean B B, Crawley J A, Schmitt C M, Wong J, Ofman J J. The burden of illness of gastro-oesophageal reflux disease: impact on work productivity. Aliment Pharmacol Ther. May 15, 2003; 17(10):1309-17; Shaker R, Castell D O, Schoenfeld P S, Spechler S J. Nighttime heartburn is an under-appreciated clinical problem that impacts sleep and daytime function: the results of a Gallup survey conducted on behalf of the American Gastroenterological Association. Am. J. Gastroenterol. July 2003; 98(7):1487-93). The development of drugs that inhibited acid production, firstly the histamine H2 receptor antagonists and then the proton pump inhibitors revolutionized the management of GERD. Proton Pump Inhibitors (PPIs) now dominate therapy of acid related gastrointestinal disease including GERD. PPIs are commonly used as monotherapy, either as once-daily or twice-daily dosing. PPIs are also used in combination with H2 receptor antagonists and antibiotics, particularly in Helicobacter positive patients.

Despite their success, PPIs have not been wholly effective in managing GERD (Tytgat G N J. Possibilities and shortcomings of maintenance therapy in gastroesophageal reflux disease. Dig Surgery 1999; 16:1-6). In particular, many patients recover acid secretion during the night even with twice-daily administration of PPIs. This phenomenon has been termed “nocturnal acid breakthrough” (NAB) and is defined as intragastric pH less than 4 for more than one hour in the overnight period (Shaker R, Castell D O, Schoenfeld P S, Spechler S J. Night-time heartburn is an under-appreciated clinical problem that impacts sleep and daytime function: the results of a Gallup survey conducted on behalf of the American Gastroenterological Association. Am J Gastroenterol. July 2003; 98(7):1487-93; Peghini P L, Katz P O, Bracy N A, Castell D O. Nocturnal recovery of gastric acid secretion with twice-daily dosing of proton pump inhibitors. Am J Gastroenterol. May 1998; 93(5):763-7). NAB can cause refractory GERD and delay esophagitis/ulcer healing. It has also been reported that the duration of NAB influences the effectiveness of H. pylori eradication (Kim J I I, Park S H, Kim J W, Chung I S, Chung K W, Sun H S. The effects of nocturnal acid breakthrough on Helicobacter pylori eradication. Helicobacter 2002; 7(6):331-336).

Nocturnal acid breakthrough (NAB) appears, according to some investigators, about 7.5 hours following an evening dose of PPI, affects about three quarters of individuals and is seen in both patients with GERD and normal volunteers (Shaker R, Castell D O, Schoenfeld P S, Spechler S J. Night-time heartburn is an under-appreciated clinical problem that impacts sleep and daytime function: the results of a Gallup survey conducted on behalf of the American Gastroenterological Association. Am J Gastroenterol. July 2003; 98(7):1487-93; Peghini P L, Katz P O, Bracy N A, Castell D O. Nocturnal recovery of gastric acid secretion with twice-daily dosing of proton pump inhibitors. Am J Gastroenterol. May 1998; 93(5):763-7). The pattern of NAB is consistent with a circadian pattern that typically begins at about midnight and extends for a number of hours thereafter (Katz P O, Anderson C, Khoury R, Castell D O. Gastro-oesophageal reflux associated with nocturnal gastric acid breakthrough on proton pump inhibitors. Aliment Pharmacol Ther. December 1998; 12(12):1231-4; Peghini P L, Katz P O, Castell D O. Ranitidine controls nocturnal gastric acid breakthrough on omeprazole: a controlled study in normal subjects. Gastroenterology. December 1998; 115(6):1335-9). In particular, the period from midnight to 3 am is particularly marked by the ‘natural’ fall in gastric pH (this is well demonstrated in FIGS. 4A, 5A, and 6A and the baseline intragastric pH parameters during this period of Example 2). While the exact mechanism of NAB is unclear, reasons could include a circadian rhythm in synthesis and processing of the proton pump, with the appearance of new pumps at night, the short half-life of PPI's and slower acid clearance at night (Hirschowitz B I, Keeling D, Lewin M, Okabe S, Parsons M, Sewing K, Wallmark B, Sachs G. Pharmacological aspects of acid secretion. Dig Dis Sci. February 1995; 40(2 Suppl):3S-23S).

Modification of the dosage regimen of PPIs has only had limited success in controlling NAB. Administration twice daily or at night are more effective than administration in the morning in that the percentage of time with gastric pH <4 is reduced but by no means eliminated (Hatlebakk J G, Katz P O, Kuo B, Castell D O. Nocturnal gastric acidity and acid breakthrough on different regimens of omeprazole 40 mg daily. Aliment Pharmacol Ther. December 1998; 12(12):1235-40; Ours T M, Fackler W K, Richter J E, Vaezi M F. Nocturnal acid breakthrough: clinical significance and correlation with esophageal acid exposure. Am J Gastroenterol. March 2003; 98(3):545-50). Adding a H₂ receptor antagonist at night-time has been found by some to reduce NAB (Xue S, Katz P O, Banerjee P, Tutuian R, Castell D O. Bedtime H₂ blockers improve nocturnal gastric acid control in GERD patients on proton pump inhibitors. Aliment Pharmacol Ther. September 2001; 15(9):1351-6), but this has been challenged (Ours T M, Fackler W K, Richter J E, Vaezi M F. Nocturnal acid breakthrough: clinical significance and correlation with esophageal acid exposure. Am J Gastroenterol. March 2003; 98(3):545-50; Orr W C, Harnish M J. The efficacy of omeprazole twice daily with supplemental H2 blockade at bedtime in the suppression of nocturnal oesophageal and gastric acidity. Aliment Pharmacol Ther. Jun. 15, 2003; 17(12):1553-8). Indeed administration of omeprazole every eight hours produced the best total gastric acid suppression but NAB still occurred (Ours T M, Fackler W K, Richter J E, Vaezi M F. Nocturnal acid breakthrough: clinical significance and correlation with esophageal acid exposure. Am J Gastroenterol. March 2003; 98(3):545-50).

In view of the above, there is still an existing need for a proton pump inhibitor formulation that can be administered as a preventive and/or therapeutic treatment of NAB, which does not require the administration of any other active ingredients, such as histamine₂ receptor antagonists, in conjunction with the proton pump inhibitor. Still further, there is still a need for a proton pump inhibitor formulation that can be administered once a day, and optionally two or more times a day, to treat NAB. Moreover, there is a need for a proton pump inhibitor formulation that has an extended release of the proton pump inhibitor in a formulation designed to treat NAB. There is a further need for an extended release formulation that will vary in its release rate with pH as the pH varies in the intestines.

### SUMMARY OF THE INVENTION

The present inventors have surprisingly discovered that proton pump inhibitors (PPIs) have a relatively narrow window of bioavailability from the gastro-intestinal tract. Without wishing to be bound by any particular theory of operation, it appears that, in addition to being degraded by the acid contents of the stomach, PPIs are primarily orally bioavailable from a discrete portion of the upper small intestine. After passing through the primary bioavailability site, the bioavailability rapidly decreases. The present invention is based on this discovery, as well as others.

The present invention provides methods of controlling stomach acid secretion in a mammal by orally administering a pharmaceutical formulation to the mammal, wherein the pharmaceutical formulation includes at least one proton pump inhibitor structured and arranged to provide an initial delayed release of a proton pump inhibitor, and a pH-dependent extended-release of a proton pump inhibitor.

The invention also provides methods of controlling stomach acid secretion in a mammal by orally administering a pharmaceutical formulation comprising from about 10 to about 60 mg of omeprazole to the mammal. In some embodiments, the pharmaceutical formulation includes a first component to provide an initial pH-dependent delayed release of omeprazole, and a second component to provide a pH-dependent extended release of the omeprazole; wherein the first component comprises: a core comprising up to about 30 mg omeprazole, and a pH-dependent coating; the second component comprises: a core comprising up to about 40 mg omeprazole, a pH-dependent coating, and a pH-dependent extended release coating. In some embodiments, the formulation includes a single component that comprises: a core comprising up to about 60 mg omeprazole, a pH-dependent coating, and a pH-dependent extended release coating.

The invention still further provides methods of controlling nocturnal acid breakthrough in a patient undergoing proton pump inhibitor therapy, the method including the steps of: identifying a patient undergoing proton pump inhibitor therapy and exhibiting symptoms of nocturnal acid breakthrough; and switching the patient from his or her current proton pump inhibitor therapy to a proton pump inhibitor therapy that comprises ingesting, once daily, in the evening, an extended release proton pump inhibitor formulation comprising a core comprising at least one proton pump inhibitor, which is coated with a pH-dependent coating, which is further coated with a pH-dependent extended release coating, wherein ingesting the extended-release proton pump inhibitor formulation results in a median-maximum plasma concentration of the proton pump inhibitor at least two hours after administration.

The invention also provides pharmaceutical formulations. Embodiments generally have at least two components: a first component exhibiting an initial release of a proton pump inhibitor, and in some embodiments, the first component includes a polymer exhibiting a pH-dependent dissolution, and in other embodiments, the first component provides immediate release of the proton pump inhibitor. Embodiments with at least two components include at least a second component exhibiting an extended release of a proton pump inhibitor, the second component comprising a polymer exhibiting a pH-dependent dissolution and further comprising at least one rate-controlling excipient, which, alone or in combination with other rate-controlling excipients, provides for a pH-dependent extended release of the proton pump inhibitor.

These and other objects, aspects, embodiments and features of the invention will become more fully apparent when read in conjunction with the following detailed description.

### BRIEF DESCRIPTION OF THE DRAWINGS

FIG. 1 shows release rates for Formulation A pellets tested at target pH values of 7.15, 7.20, and 7.25.

FIG. 2 shows the mean plasma concentration versus time profile for base Formulations A, B, and C, as compared to a reference product.

FIG. 3A shows the normal pH profile. FIG. 3B shows the pH profile with Formulation A dosed at 10 pm.

FIG. 4A shows the normal pH profile. FIG. 4B shows the pH profile with Formulation B dosed at 10 pm.

FIG. 5A shows the normal pH profile. FIG. 5B shows the pH profile with Formulation C dosed at 10 pm.

FIG. 6A shows the normal pH profile. FIG. 6B shows the pH profile with the prior art product dosed twice daily.

FIG. 7 graphically demonstrates the relationship between the Eudragit S concentration in the extended release coating and delay to maximum plasma concentration (tₘₐₓ) and lag time (t_{lag}) for omeprazole.

FIG. 8 graphically represents the mean omeprazole plasma concentration versus time, by treatment profile for a number of two component formulations (linear scale) dosed at dinner time.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments, with occasional reference to the accompanying drawings. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Throughout this disclosure, reference will be made to compounds according to the invention. Reference to such compounds, in the specification and claims, includes esters and salts of such compounds. Thus, even if not explicitly recited, such esters and salts are contemplated, and encompassed, by reference to the compounds themselves.

All percent measurements in this application, unless otherwise stated, are measured by weight based upon 100% of a given sample weight. Thus, for example, 30% represents 30 weight parts out of every 100 weight parts of the sample.

As the term is used herein, “proton pump inhibitors,” or “PPIs” relates to drugs that act to inhibit proton pumps. PPIs include, but are not limited to, compounds, derivatives of compounds, forms of compounds, such as isomers, stereoisomers, salts, hydrates, and solvates, that have activity as proton pump inhibitors. Proton pump inhibitors are potent inhibitors of gastric acid secretion, inhibiting H⁺, K⁺-ATPase, the enzyme involved in the final step of hydrogen ion production in the parietal cells. Moreover, the proton pump inhibitors typically include benzimidazole compounds. Proton pump inhibitors according to the present invention include, but are not limited to, omeprazole, lansoprazole, pantoprazole, rabeprazole, esomeprazole, leminoprazole, tenatoprazole, and their stereoisomers, enantiomers, and tautomers, and various salts thereof, such as, for example, alkaline salts. For example, and without limitation, proton pump inhibitors including various benzimidazole compounds useful in the formulations according to the present invention include those disclosed in the following documents, the disclosures of which are incorporated by reference herein in their entireties: U.S. Pat. No. 4,045,563, U.S. Pat. No. 4,255,431, U.S. Pat. No. 4,182,766, U.S. Pat. No. 4,359,465, U.S. Pat. No. 4,472,409, U.S. Pat. No. 4,508,905, U.S. Pat. No. 4,628,098, U.S. Pat. No. 4,738,975, U.S. Pat. No. 5,045,321, U.S. Pat. No. 4,786,505, U.S. Pat. No. 4,853,230, U.S. Pat. No. 5,045,552, U.S. Pat. No. 5,312,824, U.S. Pat. No. 5,877,192, U.S. Pat. No. 6,207,198, and U.S. Pat. No. 6,544,556, EP-A-0295603, EP-A-0166287, EP-A-0519365, EP-A-005129, EP-A-0174726, and GB 2,163,747.

Initially, it should be noted that the terms “controlled,” “extended,” and “sustained,” when used to describe a release profile for a PPI drug, are used interchangeably herein. The term “extended release” is intended to encompass controlled, extended, and sustained release. Thus, “extended release” encompasses any rate of release that is not immediate or delayed immediate (delayed release).

“Delayed release” describes a formulation that begins to release after some period of delay, during which essentially no drug is released. The release that occurs after the delay is immediate, i.e., >75% at 30 minutes.

“Immediate release” describes a formulation that releases the drug upon dissolution, without significant delay. In most embodiments, such formulations would release drug in the upper GI, including the mouth, esophagus, and/or stomach.

The term “rate-controlling excipient” is intended to encompass the variety of excipients that may be included in a formulation to control the rate of release of the PPI drug from the formulation. Rate-controlling excipients include, but are not limited to, polymers, which may exhibit varying degrees of water solubility and varying degrees of pH-dependence in their solubility. Rate-controlling excipients also include additional elements that may, in some instances, be combined with polymers or other components to control a rate of drug release. Examples of such rate-controlling excipients include, but are not limited to, talc.

Also, “coatings” are described herein in reference to a layer of a composition that is applied to a substrate, which may be a core or may be a previous coating. During in vivo use, or when hydrated, coatings can become “membranes,” which is a term that attempts to describe how the coating functions when hydrated. Thus, “coating” and “membrane” may be used interchangeably herein and unless the context clearly indicates otherwise, no distinction is intended.

It is well recognized that proton pump inhibitors (PPIs) are acid labile (typically a first order degradation). Thus, in order to maintain maximum efficacy, it is important to prevent the PPIs from being degraded by the contents of the stomach. Enteric coating technology is preferred, as it dissolves only upon reaching a less acidic pH (typically about pH 5.5), and thus, enteric-coated PPI formulations that then rapidly release the drug should theoretically protect the PPI from being degraded by the acid contents of the stomach and limit the amount of degradation in the intestines. Alternatively, immediate release formulations of PPIs may be prepared so as to buffer, or raise the pH of, the contents of the stomach, thereby allowing the release of the PPI directly into the stomach.

Most, if not all, of the commercially available PPI products include an enteric coating to prevent the acid labile drug from being exposed to the contents of the acid of the stomach. Beyond the enteric coating needed to prevent premature release of the PPI, however, commercially available PPI formulations have no mechanism to provide for an extended release of the drug product. Thus, these formulations provide an initial bolus release of the drug, which provides a sharp spike in plasma concentration of the drug, which is followed by a decrease in plasma concentration as the drug is eliminated.

The problem, as noted in the Background section above, is that the proton pumps in the stomach become active again after the absorbed PPI has been metabolized. If this occurs during waking hours, it may be addressed at the time by taking another PPI or an antacid or another form of treatment. Often, however, it occurs during sleeping hours and the “nocturnal acid breakthrough” results in middle-of-the night heartburn. Thus, there is a need in the art for a PPI formulation that can provide relief during waking hours and also prevent the occurrence of nocturnal acid breakthrough.

The present inventors have surprisingly discovered that proton pump inhibitors have a window of bioavailability from the gastro-intestinal tract. It appears that, in addition to being degraded by the acid contents of the stomach, and to an extent, also the contents of the intestines, PPIs are primarily orally bioavailable from a discrete portion of the upper intestine. After passing through the primary bioavailability site, the bioavailability of the PPI rapidly decreases, which may be a result of significant hepatic first-pass metabolism. Thus, formulations that effectively control both daytime and nighttime stomach acid should balance a desirable bioavailability, while at the same time extending the time-course of drug release. The present invention provides such formulations.

It has also been appreciated by the inventors that care should be taken to prepare the formulations to minimize the degradation of the PPI in the intestines as it is released from an extended release dosage form. That is, while the pH rises to above 5.5 relatively quickly posterior to the stomach, the subsequent gradient of increasing pH up to about 7.25 can continue to degrade the PPI, thereby further reducing bioavailability. Thus, in some embodiments of the invention, rate-controlling excipients, which begin to dissolve only upon reaching pH 7 or higher, are used.

Embodiments according to the present invention include at least one component exhibiting an extended release of a proton pump inhibitor. The extended release component may comprise a polymer exhibiting a pH-dependent dissolution characteristic and further comprise at least one rate-controlling excipient that provides for a pH-dependent extended release of the PPI. In some embodiments, this is an extended release that varies the release rate according to the pH, particularly exhibiting a slower release rate (and thus exposing less PPI to degradation) at pH less than 7 while exhibiting faster release rates at higher pH values, i.e. pH 7.2, and therefore achieving essentially complete release (e.g., 80%) at a time point such as 8 hours.

The invention provides, in some embodiments, pharmaceutical formulations comprising at least two components: 1) at least a first delayed release component exhibiting an initial release of a proton pump inhibitor, the first component comprising a polymer exhibiting a pH-dependent dissolution characteristic, and/or a first component exhibiting an immediate release of a proton pump inhibitor; and 2) at least a second component, which is an extended release component exhibiting an extended release of a proton pump inhibitor, the second component comprising a polymer exhibiting a pH-dependent dissolution characteristic and further comprising at least one rate-controlling excipient that provides for a pH-dependent extended release of the PPI. In some embodiments, the second component provides an extended release that varies the release rate according to the pH, particularly exhibiting a slower release rate (and thus exposing less PPI to degradation) at pH less than 7 while exhibiting faster release rates at higher pH values, i.e. pH 7.2, and achieving essentially complete release (e.g., 80%) at a time point such as 8 hours.

The first, or delayed release, component can be structured in a variety of manners. The proton pump inhibitor may be compressed to form a core, which is coated with a polymer having a pH-dependent, e.g., “enteric,” polymer. Alternatively, a nonpareil can be spray coated with a PPI, followed by enteric coating. Immediate release components can be formulated with agents to adjust the local pH around the PPI, to reduce the likelihood of PPI degradation upon exposure to stomach contents. The choices are not limited, and are left to the practitioner. In any event, the first component will release into the stomach, and/or delay release of the PPI until only just passing the stomach and entering the upper GI, where the pH increases.

The second, or extended release, component(s) can be structured in a variety of manners as well. The proton pump inhibitor may be compressed to form a core, or alternatively, a nonpareil can be spray coated with a PPI. It has been found that an enteric coating on the second, or extended release, component(s) is desirable in preventing unwanted premature degradation of the formulation as it passes through the stomach. Thus, in some embodiments, the second, or extended release, component(s) also includes an enteric coating.

The choice of the combination of rate-controlling excipients to provide for the extended delivery of the PPI from the extended release components is not limited in any way. Examples of possible rate-controlling excipients are provided in more detail below. Again, the choices are not limited, and are left to the practitioner.

Depending on the particular need, the inventive formulations may be prepared as tablets, pellets, minitablets, caplets, or any other desired form. The particular form depends upon the desired end use and the choice is left to the practitioner. Pellet dosage forms can be, for example, encapsulated, prepared as a tablet, or administered in a food or drink. One of the advantages of encapsulated pelleted products is that the onset of absorption is generally less sensitive to stomach emptying. The entrance of the pellets into the small intestine can be more uniform than with non-disintegrating extended-release tablet formulations.

It should be noted that while multi-pellet formulations are exemplified herein, monolithic formulations are also expressly contemplated. Thus, for example, a tablet may be enterically coated, under which is layered the delayed release PPI component of the system, which provides for a PPI plasma concentration after the dosage leaves the stomach. Beneath the initial PPI layer is at least one rate-controlling excipient, under or within which is the extended release PPI component, which is released over an extended period. A monolithic form such as this is expressly contemplated and can provide the desired plasma concentrations disclosed herein as well as relief from nocturnal acid breakthrough.

Hybrid products are also expressly contemplated. For example, a multi-pellet formulation may be compressed to form a monolithic core, which is coated by an outer PPI layer followed by an enteric coating. Alternatively, a polymeric capsule containing a multi-pellet formulation may itself be enterically coated. The choice of the particular combination and the means for achieving the plasma drug levels are not critical.

Turning to a more detailed discussion of the structure of some embodiments, a delayed release component (when present) includes a PPI core and a pH-dependent coating; and an extended release component comprises: a core comprising at least one proton pump inhibitor, a pH-dependent coating, and at least one rate-controlling excipient to provide for a pH-dependent extended release. Again, as noted above, the manner in which the delayed and extended release are achieved are not critical. Generally, the delayed release of the first component will be achieved through the use of an enteric polymer. Additionally, it may be preferred to include an enteric polymer in the other components as well.

In some embodiments of the invention, the enteric coatings are applied directly to the proton pump inhibitor, and extended release coatings can then be applied thereto. In such embodiments, there is no need for a coating to separate the PPI from the enteric coating.

Polymers that exhibit a pH-dependent dissolution are commonly used to prevent dissolution of a drug product in the acid environment around the stomach (and are commonly referred to as enteric polymers). Generally, as used herein, the term pH-dependent dissolution refers to a polymer (or other rate-controlling excipient) that exhibits a rate of dissolution that varies depending on pH. Typically, the dissolution rate of such components will be relatively low or non-existent below a particular pH (e.g., pH 5.5 in the case of Eudragit L, or pH 7.0 in the case of Eudragit S), and the dissolution rate will be rapid at pH values above the critical values (e.g. >5.5 or >7, respectively). Polymers exhibiting the desired delayed release characteristics include those dissolving at pH less than 6, and/or pH greater than 5, such as from pH 5-6. Polymers exhibiting the desired extended release characteristics include those dissolving at pH less than 7.5, and/or pH greater than 6.5, such as from pH 6.5-7.5.

Polymers exhibiting these types of dissolution characteristics are commonly referred to as “enteric” polymers and are used to form “enteric” coatings. Enteric coatings may comprise, for example, rate-controlling excipients such as cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methyl acrylate-methacrylic acid copolymer, methacrylate-methacrylic acid-octyl acrylate copolymer, etc. These may be used either alone or in combination, or together with other rate-controlling excipients than those mentioned above. The enteric coating may also include rate-controlling excipients that are neither decomposed nor solubilized in living bodies, such as alkyl cellulose derivatives such as ethyl cellulose, crosslinked polymers such as styrene-divinylbenzene copolymer, polysaccharides having hydroxyl groups such as dextran, cellulose derivatives which are treated with bifunctional crosslinking agents such as epichlorohydrin, dichlorohydrin, 1-, 2-, 3-, 4-diepoxybutane, etc.

Turning to the extended release component(s) of the formulations of the invention, it is noted that any number of methods, examples of which are known in the art, may achieve the pH-dependent extended release. Examples of extended release systems include but are not limited to, diffusion-controlled, matrix, osmotic, and ionic exchange systems. As noted above, these can be used in single-unit (monolithic) or multiunit dosage forms. With diffusion-controlled extended release systems, the formulation containing the active substance of interest, i.e., the PPI, may be surrounded by a semi-permeable membrane. Semi-permeable membranes include those that are permeable to a greater or lesser extent to both water and solute. This membrane may include water-insoluble and/or water-soluble polymers, and may exhibit pH-dependent and/or pH-independent solubility characteristics. Polymers of these types are described in detail below. Generally, the characteristics of the membrane (e.g., the composition of the membrane) will determine the nature of release from the dosage form.

In an osmotic-release system, a selectively permeable membrane encloses a reservoir of the substance of interest, i.e., the PPI, at a concentration sufficient to provide an osmotic pressure above a threshold level. Selectively permeable membranes include those that are permeable to water but not to solute. The pore or orifice size of a selectively permeable membrane can be varied so that passage of molecules of the substance through the pore or orifice of the membrane becomes the rate-limiting factor in dispensing the substance into the surrounding environment outside of the dosage form. Alternatively, the reservoir of the substance, in addition to the active ingredient, may also include an inactive substance, such as an osmotic agent, which is present at a concentration sufficient to provide an osmotic pressure above a threshold level.

Matrix-type systems comprise an active substance of interest, i.e., the PPI, mixed with, for example, water-soluble, e.g., hydrophilic polymers, or water-insoluble, e.g., hydrophobic polymers, and can exhibit a dissolution that is pH-independent or pH-dependent. Generally, the properties of the polymer used in a extended-release system will affect the mechanism of release. For example, the release of the active ingredient from a system containing a hydrophilic polymer can proceed via both surface diffusion and/or erosion. Mechanisms of release from pharmaceutical systems are well known to those skilled in the art. Matrix-type systems can be used in monolithic or multi-unit, and may be coated with water-soluble and/or water-insoluble polymeric membranes, examples of which are described herein.

The extended release component of the inventive formulations and methods may rely on ion exchange resins for the release of the PPI. In such formulations, the drug is bound to ion exchange resins and, when ingested, the release of drug can be determined by the ionic environment within the gastrointestinal tract. Note that all of the components involved in controlling the rate of release of the PPI drug from the formulation, including components of diffusion, matrix, osmotic, and ionic, are considered “rate-controlling excipients.”

The release of the PPI can be modified or controlled by using, for example, polymers in varying proportions to provide the desired release profile, other polymer matrices, gels, permeable membranes, osmotic systems, multilayer coatings, microparticles, liposomes, microspheres, or the like, or combinations thereof. Examples of suitable extended release formulations are known to those of ordinary skill in the art, and may readily be selected for use with the PPI compositions of the present invention. Thus, tablets, capsules, gelcaps, caplets, and the like, that are adapted for extended-release, may be used in accordance with the presently disclosed methods. The extended-release of the active ingredient may be triggered or stimulated by various inducers, such as, for example, pH, temperature, enzymes, water, and/or other physiological conditions or compounds.

As generally discussed above, the formulations of the present invention generally comprise at least one polymeric material, which may be used in an enteric coating providing for a delayed release, or may be used in an extended release coating providing for a controlled rate of release. As noted above, such polymers may be primarily water-soluble or primarily water-insoluble. Polymers are generally used in the present formulations as rate-controlling excipients.

Suitable water-soluble polymers include, but are not limited to, polyvinyl alcohol, polyvinylpyrrolidone, methylcellulose, hydroxypropylcellulose, hydroxypropylmethyl cellulose, or polyethylene glycol, and/or mixtures thereof.

Suitable water-insoluble polymers include, but are not limited to, ethylcellulose, cellulose acetate cellulose propionate, cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), low density poly(ethylene), high density poly(ethylene), poly(ethylene oxide), poly(ethylene terephthalate), poly(vinyl isobutyl ether), poly(vinyl acetate), poly(vinyl chloride) or polyurethane, and/or mixtures thereof.

EUDRAGIT™ polymers (available from Rohm Pharma) are polymeric lacquer substances based on acrylates and/or methacrylates. A suitable polymer that is freely permeable to the active ingredient and water is EUDRAGIT™ RL. A suitable polymer that is slightly permeable to the active ingredient and water is EUDRAGIT™ RS.

EUDRAGIT™ RL and RS are acrylic resins comprising copolymers of acrylic and methacrylic acid esters with a low content of quaternary ammonium groups. The ammonium groups are present as salts and give rise to the permeability of the lacquer films. EUDRAGIT™ RL and RS are freely permeable (RL) and slightly permeable (RS), respectively, independent of pH. The polymers swell in water and digestive juices, in a pH-independent manner. In the swollen state, they are permeable to water and to dissolved active compounds.

EUDRAGIT™ L is an anionic polymer synthesized from methacrylic acid and methacrylic acid methyl ester, and is insoluble in acids. The solubility of EUDRAGIT™ L is pH dependent. Above about pH 5.5, the polymer becomes soluble.

EUDRAGIT™ S is an anionic polymer synthesized from methacrylic acid and methacrylic acid methyl ester, and is insoluble in acids. The solubility of EUDRAGIT™ S is pH dependent. Above about pH 7.0, the polymer becomes soluble.

In one embodiment, the polymeric material comprises methacrylic acid co-polymers, ammonio methacrylate co-polymers, or mixtures thereof. Methacrylic acid co-polymers such as EUDRAGIT™ S and EUDRAGIT™ L (Rohm Pharma) are also suitable for use in the controlled release formulations of the present invention. These polymers are gastroresistant and enterosoluble polymers. The polymer films are insoluble in pure water and diluted acids. They dissolve at higher pHs, depending on their content of carboxylic acid. EUDRAGIT™ S and EUDRAGIT™ L can be used as single components in the polymer coating or in combination in any ratio. By using a combination of the polymers, the polymeric material may exhibit a solubility at a pH between the pHs at which EUDRAGIT™ L and EUDRAGIT™ S are separately soluble.

Ammonio methacrylate co-polymers such as EUDRAGIT™ RS and EUDRAGIT™ RL (Rohm Pharma) are also suitable for use in the controlled release formulations of the present invention. These polymers are insoluble in pure water, dilute acids, buffer solutions, or digestive fluids over the entire physiological pH range. The polymers swell in water (and digestive fluids independently of pH). In the swollen state, they are then permeable to water and dissolved actives. The permeability of the polymers depends on the molar ratio of ethylacrylate (EA), methyl methacrylate (MMA), and trimethylammonioethyl methacrylate chloride (TAMCI) groups in the polymer. Those polymers having EA:MMA:TAMCI ratios of 1:2:0.2 (EUDRAGIT™ RL) are more permeable than those with ratios of 1:2:0.1 (EUDRAGIT™ RS). Polymers of EUDRAGIT™ RL are insoluble polymers of high permeability. Polymers of EUDRAGIT™ RS are insoluble films of low permeability.

The ammonio methacrylate co-polymers may be combined in any desired ratio. For example, a weight ratio of EUDRAGIT™ RS:EUDRAGIT™ RL (90:10) may be used. The ratios may be adjusted to provide a delay in release of the drug. For example, the weight ratio of EUDRAGIT™ RS:EUDRAGIT™ RL may be about 100:0 to about 80:20, about 100:0 to about 90:10, or any weight ratio in between. In such formulations, the less permeable polymer EUDRAGIT™ RS would generally comprise the majority of the polymeric material.

The ammonio methacrylate co-polymers may be combined with the methacrylic acid co-polymers within the polymeric material in order to achieve the desired delay in release of the drug. Weight ratios of ammonio methacrylate co-polymer (e.g., EUDRAGIT™ RS) to methacrylic acid co-polymer in the range of about 99:1 to about 20:80 may be used. The two types of polymers can also be combined into the same polymeric material, or provided as separate coats that are applied to the core.

In addition to the EUDRAGIT™ polymers described above, a number of other such copolymers may be used to create a delay in drug release. These include methacrylate ester co-polymers (e.g., EUDRAGIT™ NE 30D). Further information on the EUDRAGIT™ polymers is to be found in “Chemistry and Application Properties of Polymethacrylate Coating Systems”, in Aqueous Polymeric Coatings for Pharmaceutical Dosage Forms, ed. James McGinity, Marcel Dekker Inc., New York, pg 109-114).

The rate of PPI release (delayed or extended) may be achieved by systems comprising a polymeric material comprising a major proportion (i.e., greater than 50% of the total polymeric content) of one or more pharmaceutically acceptable water-soluble polymers, and optionally a minor proportion (i.e., less than 50% of the total polymeric content) of one or more pharmaceutically acceptable water-insoluble polymers.

Alternatively, rate of PPI release (delayed or extended) may be achieved by systems comprising a major proportion (i.e., greater than 50% of the total polymeric content) of one or more pharmaceutically acceptable water insoluble polymers, and optionally a minor proportion (i.e., less than 50% of the total polymeric content) of one or more pharmaceutically acceptable water-soluble polymers.

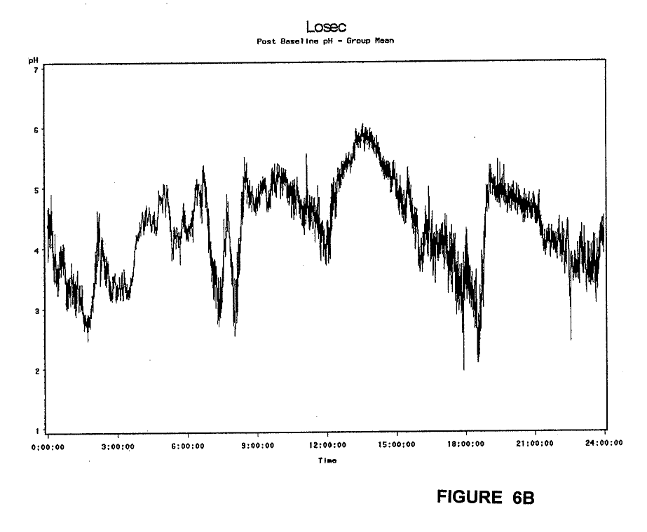

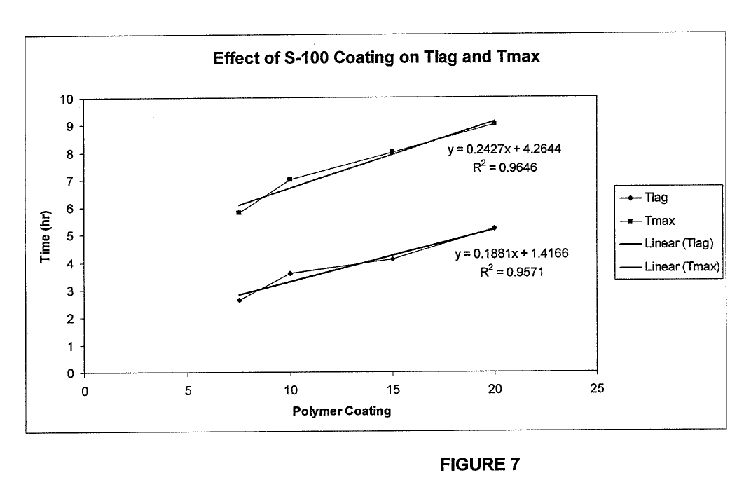

The amount of polymer to be used in an extended release system is typically adjusted to achieve the desired drug delivery properties, including the amount of drug to be delivered, that rate and location of drug delivery, the time delay of drug release, and the size of the multiparticulates in the formulation. In a multiparticulate formulation, the amount of polymer applied typically constitutes about 1 to about 50 wt % of the formulation. In some embodiments, the amount of polymer in the formulation, as a weight percent of the entire formulation, can range from 2 to about 20 wt %, or from 5 to about 15 wt %, or from about 7.5 to about 12.5 wt %, or about 10 wt %. Of course, the amount of polymer used will depend upon the particular polymer choice. A graphical presentation of the time to maximum plasma concentration, compared to the amount of Eudragit S in a particular formulation, is presented in FIG. 7.

The formulations used in the present methods may include any number of pharmaceutically acceptable excipients. Suitable excipients include, but are not limited to, carriers, such as sodium citrate or dicalcium phosphate; fillers or extenders, such as stearates, silicas, gypsum, starches, lactose, sucrose, glucose, mannitol, talc, or silicic acid; binders, such as hydroxymethyl-cellulose, alginates, gelatin, polyvinyl pyrrolidone, sucrose or acacia; humectants, such as glycerol; disintegrating agents, such as agar, calcium carbonate, potato or tapioca starch, alginic acid, certain silicates, or sodium carbonate; solution retarding agents, such as paraffin; absorption accelerators, such as quaternary ammonium compounds; wetting agents, such as cetyl alcohol or glycerol monostearate; absorbents, such as kaolin and bentonite clay; lubricants, such as talc, calcium stearate, magnesium stearate, solid polyethylene glycols, and sodium lauryl sulfate; stabilizers, such as fumaric acid; coloring agents; buffering agents; dispersing agents; preservatives; organic acids; and organic bases.

The aforementioned excipients are given as examples only and are not meant to include all possible choices. Additionally, many excipients may have more than one role, or be classified in more than one group; the classifications are descriptive only, and not intended to limit any use of a particular excipient. Thus, for example, talc can be used as a lubricant to facilitate processing, or it may be formulated with a polymer system to act as a rate-controlling excipient.

Examples of suitable organic acids include, but are not limited to, adipic acid, ascorbic acid, citric acid, fumaric acid, malic acid, succinic acid, tartaric acid, and mixtures thereof. Suitable organic bases, include, but are not limited to, sodium citrate, sodium succinate, sodium tartrate, potassium citrate, potassium tartrate, potassium succinate, and mixtures thereof. Alkalinizing agents and basifying agents are expressly contemplated. Suitable diluents include, but are not limited to, lactose, talc, microcrystalline cellulose, sorbitol, mannitol, xylitol, fumed silica, stearic acid, magnesium stearate, sodium stearate, and mixtures thereof.

As noted above, in some embodiments of the invention, the polymeric material itself is formulated with one or more soluble excipients so as to increase the permeability of the polymeric material. The soluble excipient may be selected from among, for example, soluble polymers, surfactants, alkali metal salts, organic acids, sugars, and sugar alcohols. Such soluble excipients include, but are not limited to, polyvinyl pyrrolidone, polyethylene glycol, sodium chloride, surfactants such as sodium lauryl sulfate and polysorbates, organic acids such as acetic acid, adipic acid, citric acid, fumaric acid, glutaric acid, malic acid, succinic acid, and tartaric acid and sugars such as dextrose, fructose, glucose, lactose and sucrose, and sugar alcohols such as lactitol, maltitol, mannitol, sorbitol and xylitol, xanthan gum, dextrins, and maltodextrins. In some particular embodiments, polyvinyl pyrrolidone, mannitol and/or polyethylene glycol are the soluble excipients. The soluble excipient is typically used in an amount of from about 1% to about 10% by weight, based on the total dry weight of the polymer. Because these components generally act to increase the permeability of the polymer systems with which they are formulated, they are generally considered “rate-controlling excipients.”

The polymeric material can also include one or more auxiliary agents such as fillers, plasticizers, and/or anti-foaming agents. Any of these agents may be included to improve processing or to modify the qualities of the end product.

Representative fillers include talc, fumed silica, glyceryl monostearate, magnesium stearate, calcium stearate, kaolin, colloidal silica, gypsum, micronized silica, and magnesium trisilicate. The quantity of filler used typically ranges from about 2% to about 300% by weight, and can range from about 20 to about 100%, based on the total dry weight of the polymer. In one embodiment, talc is the filler.

Plasticizers include, but are not limited to, for example, adipates, azelates, benzoates, citrates, isoebucates, phthalates, sebacates, stearates, and glycols. Representative plasticizers include, for example, acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, gylcerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-i-octyl phthalate, di-i-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40, or 50%, based on the weight of the dry polymer.

In one embodiment, the anti-foaming agent is simethicone. The amount of anti-foaming agent used typically comprises from about 0% to about 0.5% of the final formulation.

The combination of all solid components of the polymeric material, including co-polymers, fillers, plasticizers, and optional excipients and processing aids, typically provides an about 10 to about 450% weight gain on the cores. In one embodiment, the weight gain is about 30 to about 160%.

The polymeric material can be applied by any known method, for example, by spraying using a fluidized bed coater (e.g., Wurster coating) or pan coating system. In one embodiment, the formulations of the present invention are provided as multiparticulate formulations. In some embodiments, the PPI is formed into an active core by applying the compound to a nonpareil seed having an average diameter in the range of about 0.4 to about 1.1 mm or about 0.85 to about 1.00 mm. The PPI may be applied with or without additional excipients (detailed below) onto the inert cores, and may be sprayed from solution or suspension using a fluidized bed coater (e.g., Wurster coating) or pan coating system. Alternatively, the PPI may be applied as a powder onto the inert cores using a binder to bind it onto the cores. Active cores may also be formed by extrusion of the core with suitable plasticizers and any other processing aids as necessary.

Components of the invention may be dried or cured after application of the polymeric material. Curing means that the multiparticulates are held at a controlled temperature for a time sufficient to provide stable release rates. Curing can be performed for example in an oven or in a fluid bed drier. Curing can be carried out at any temperature above room temperature.

A sealant or barrier can be applied to a polymeric coating. A sealant or barrier layer may also be applied to a core prior to applying a polymeric material. The sealant or barrier layer generally does not modify the release of the PPI significantly. Suitable sealants or barriers are permeable or soluble agents such as hydroxypropyl methylcellulose, hydroxypropyl cellulose, hydroxypropyl ethylcellulose, and xanthan gum. Hydroxypropyl methylcellulose is particularly useful in this regard.

Other agents can be added to improve the processability of a sealant or barrier layer. Such agents include talc, colloidal silica, polyvinyl alcohol, titanium dioxide, micronized silica, fumed silica, glycerol monostearate, magnesium trisilicate or magnesium stearate or a mixture thereof. The sealant or barrier layer can be applied from solution (e.g., aqueous) or suspension using any known means, such as a fluidized bed coater (e.g., Wurster coating) or pan coating system. Suitable sealants or barriers include, for example, OPADRY WHITE Y-1-7000 and OPADRY OY/B/28920 WHITE, each of which is available from Colorcon Limited, England.

The invention also provides an oral dosage form containing a multiparticulate, PPI formulation as hereinabove defined, in the form of caplets, capsules, particles for suspension prior to dosing, sachets, or tablets. When the dosage form is in the form of tablets, the tablets may be, for example, disintegrating tablets, fast dissolving tablets, effervescent tablets, fast melt tablets, and/or mini-tablets. The dosage form can be of any shape suitable for oral administration of a drug, such as spheroidal, cube-shaped oval, or ellipsoidal. The dosage forms will generally be prepared in a manner known in the art and include addition pharmaceutically acceptable excipients, as desired.

As shown above, the formulations of the invention can achieve the desired plasma levels in a variety of manners. These formulations may deliver the PPI at a variety of rates and still achieve the desired plasma concentrations. For example, in some embodiments, the extended release formulation or the ER component exhibits the following rate of release of the proton pump inhibitor, when tested in a USP Type I and II dissolution test apparatus in pH 1.2 for two hours (in Type I apparatus) followed by pH 6.8 for the remainder of the test, or followed by switching to pH 7.2 after two hours in pH 1.2 (in Type I apparatus). Details of the test are provided in the Examples section below; when dissolution testing is referred to herein, tests are performed according to the detailed description in the Examples section below. It will be appreciated that other test conditions may be used including, for example, USP Type IlIl apparatus with change of pH medium from strongly acid (i.e., pH 1.2) to intestinal (i.e., pH 6.5-6.8) to lower intestinal/colon (pH about 7.2).

All of the extended release formulations or extended release components of the invention release substantially no drug during the two-hour testing at pH 1.2. Generally, this is less than about 5%. When tested at pH 6.8, following two hours at pH 1.2, the following results are observed: at 30 minutes: less than about 20% released; and one hour: less than about 50% released. More preferably, at 30 minutes: less than about 10% released; and one hour: less than about 20% released.

The same extended release formulations or extended release components, when tested at pH 7.2, following two hours at pH 1.2, the following results are observed: at six hours: greater than about 50% is released; eight hours: greater than about 70% released; and 12 hours: greater than about 80% released.

The present invention also provides treatments that achieve particular plasma drug levels at particular hours after administration. In some embodiments, those plasma drug levels are achieved by the formulations having the release rates described above. In some embodiments, for example, a formulation comprises from about 10 to about 60 mg omeprazole, and orally administering the formulation produces a maximum plasma concentration of omeprazole at greater than two hours after administration, such as greater than or equal to four hours after administration. In some embodiments, the maximum plasma concentration is achieved at a time between two and twelve hours after administration, or between two and eight hours after administration. The presence of food in the stomach may extend the time to achieve the maximum plasma concentration.

The therapeutic effects, plasma levels, and/or release rates described herein can be achieved in a variety of manners. In one two-component embodiment according to the invention, the delayed release component comprises from about 25 to about 90% of the entire dose (with the remaining about 75 to about 50% being in the extended release component). More particularly, the delayed or immediate release component will comprise 25, 30, 35, 40, 45, or 50% of the entire dose. In some particular embodiments, the formulation comprises from about 10 to about 60 mg omeprazole, wherein the first (e.g., delayed release) component comprises up to about 30 mg of omeprazole and the second (i.e., extended release) component comprises up to about 40 mg of omeprazole. In one particular embodiment, the first component comprises 10 mg omeprazole and the second component comprises 30; in another particular embodiment, the first comprises 20 mg omeprazole and the second comprises 20 mg omeprazole.

In one embodiment, the PPI formulations initially delay the release of the drug by the use of an enteric coating. Following the delay, the formulation may rapidly release the drug, followed by extending the release for a specified period. The extended release over time is useful, for example, to provide a subject with therapeutic drug levels in the early morning hours following an evening (e.g., dinnertime) administration, during which time NAB would normally occur. As a result, a subject can take the drug at night prior to sleep, and obtain the therapeutic benefits during sleeping hours. This is particularly useful in treating, preventing, and/or managing nocturnal acid breakthrough, and associated pathologies.

In addition to, or instead of, other components described herein, the formulations may include a component that immediately releases the PPI soon after administration, i.e., without any delay in the release. Such formulations would provide a rapid and/or immediate therapeutic effect for the subject.

The amount of the dose administered, as well as the dose frequency, will vary depending on the particular dosage form used and route of administration. The amount and frequency of administration will also vary according to the age, body weight, and response of the individual subject. A competent physician can readily determine typical dosing regimens without undue experimentation. It is also noted that the clinician or treating physician will know how and when to interrupt, adjust, or terminate therapy in conjunction with individual subject response.

In general, the total daily dosage for treating, preventing, and/or managing the GI conditions described herein will depend upon the particular PPI used. For omeprazole, the amount is from about 5 mg to about 360 mg, or from about 10 mg to about 120 mg, or from about 20 mg to about 80 mg, or from about 20 mg to about 40 mg of omeprazole, or a pharmaceutically acceptable salt thereof. Other PPIs can be used, and are generally used in an amount that produces an effect on proton pump inhibition that is roughly equivalent to that inhibition produced by omeprazole in the amounts listed above. Acceptable dosage ranges for most PPIs have been established and published elsewhere.

The pharmaceutical compositions containing the PPI may be administered once every 1, 2, 3, 4, 5, or more days. In one embodiment, the pharmaceutical compositions are administered once per day.

In some once-daily embodiments of the invention, the drug is administered in the morning, and in some once-daily embodiments of the invention, the drug is administered in the evening. While the timing of administration, in relation to mealtime, is not critical, it may be advantageous to administer the drug immediately prior to breakfast or prior to eating dinner in the evening. Such timing can, in some embodiments, align the delivery of the drug with the natural stomach acid secretion. Preferably, the formulation is administered within one hour, and more preferably, within thirty minutes, prior to or following the eating of an evening meal, such as dinner, typically at around 6 pm. In clock hours, the formulations of the invention will generally be administered from about 5 PM to about 8 PM, or from about 6 PM to about 7 PM.

It should be noted that any of the pharmaceutical compositions and dosage forms described herein may further comprise one or more pharmaceutically active compounds other than a PPI. Such compounds may be included to treat, prevent, and/or manage the same condition being treated, prevented, and/or managed with the PPI, or a different one. For example, those of skill in the art are familiar with examples of the techniques for incorporating additional active ingredients into compositions comprising PPI. Alternatively, such additional pharmaceutical compounds may be provided in a separate formulation and co-administered to a subject along with a PPI composition according to the present invention. Such separate formulations may be administered before, after, or simultaneously with the administration of the PPI compositions of the present invention. In one embodiment, the PPI formulation comprises and/or is co-administered with one or more other compounds including, but not limited to, histamine-2 antagonists, antacids, antibiotics (including but not limited to clarithromycin and amoxicillin), steroids, opioids, non-steroidal anti-inflammatory agents (i.e., “NSAIDS”), including but not limited to, naproxen, ibuprofen, etc. It is noted, however, that antacids may increase the local pH in or around the dosage form and this should be considered when using an enteric polymer, which is sensitive to pH.

The present invention is useful in the treatment of a variety of diseases and conditions. Such treatments include but are not limited to, treatment of ulcer, such as duodenal or gastric ulcer, including ulcer associated with Helicobacter pylori infection, as well as treatment of such infections. Other treatments include treatment of gastroesophageal reflux disease (GERD), such as symptomatic GERD, treatment of heartburn and other symptoms associated with GERD, treatment of erosive esophagitis, as well as maintenance of the healing of erosive esophagitis. The present invention can also be used in the treatment of pathological hypersecretory conditions, such as Zollinger-Ellison syndrome, multiple endocrine adenomas, and systemic mastocytosis. Embodiments of the present invention are particularly useful in the treatment of nocturnal acid breakthrough, occurring in the use of conventional proton pump inhibitor formulations for the treatment of any of the above-identified conditions. It should also be noted that while treatment of all of the above-identified conditions is a utility of the present invention, so is the prevention of such diseases, conditions, and symptoms.

The invention is further illustrated by reference to the following examples. It will be apparent to those skilled in the art that many modifications, both to the materials and methods, may be practiced without departing from the purpose and scope of the invention.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

### EXAMPLES

### Example 1

### Preparation of Base Formulations and in Vitro Performance

This Example describes the preparation of three “base” formulations, which are tested in Example 2, and which are then used as the extended release component in various combinations in Example 3. The formulations were designed as “fast” (Formulation A), “medium” (Formulation B), and “slow” (Formulation C) release. Note that the term “fast” is used in a relative sense here, and does not necessarily mean that the formulation released quickly. Formulation A was designed to release the most quickly, Formulation B more slowly, and Formulation C the slowest of the three.

The compositions of Formulations A, B, and C are summarized in Table I below:

TABLE I

| | | | |
|---|---|---|---|
| Ingredients | Form. A | Form. B | Form. C |

Eudragit L-Coated Cores

| | | | |
|---|---|---|---|
| Omeprazole | 20.00 | 20.00 | 20.00 |
| Sugar Spheres (1.00-1.18 mm) | 127.11 | 127.11 | 127.11 |
| Sodium Lauryl sulfate (sol. 2.5%) | 0.0052 | 0.0052 | 0.0052 |
| Anhydrous Disodium Phosphate | 0.61 | 0.61 | 0.61 |
| Mannitol | 7.91 | 7.91 | 7.91 |
| Hypromellose | 16.90 | 16.90 | 16.90 |
| Macrogol 6000 | 4.02 | 4.02 | 4.02 |
| Talc | 12.27 | 12.27 | 12.27 |
| Polysorbate 80 | 1.77 | 1.77 | 1.77 |
| Titanium Dioxide | 4.02 | 4.02 | 4.02 |
| Methacrylic Acid-Ethyl | 37.40 | 37.40 | 37.40 |
| Acrylate Copolymer | | | |
| (Eudragit ® L30-D55) | | | |

Eudragit S Coating

| | | | |
|---|---|---|---|
| Eudragit S | 23.20 | 34.80 | 46.40 |
| Talc | 11.60 | 17.40 | 23.20 |
| Dibutyl Sebacate | 4.60 | 6.90 | 9.20 |
| Eudragit S Coating Subtotal | 39.40 | 59.10 | 78.80 |
| Total | 271.42 | 291.12 | 310.82 |

The Formulations were prepared as follows. Eudragit L-coated cores were purchased from Liconsa S.A. (Guadalajara, Spain). Note that such cores comprise omeprazole and include an enteric coating of Eudragit L, which, as described above, exhibits a dissolution that increases rapidly above approximately pH 5.5, e.g., pH 6.8 (typically releasing >75% at 30 minutes). Note also that these coated cores are used in Example 3 below as the delayed release component.

A Eudragit S solution was prepared as follows: 300 g of purified water and 4262.5 g of isopropyl alcohol were stirred together for 5 min.; 125 g of dibutyl sebacate was added and the mixture stirred for 5 min.; 312.5 g of talc USP is added to the mixture and stirred for 15 minutes; 5000 g of Eudragit S 12.5 (solution of methacrylate copolymer from Rohm Pharma, Germany) was added and stirred for 30 min. Note that Eudragit S also exhibits a dissolution that is dependent on pH, but the addition of talc to the coating produces an extended drug release profile.

The resulting solution was sprayed onto the Eudragit L-coated cores (Liconsa S.A.), using a fluid bed apparatus (GPCG-3, Glaft) using Wurster coating. Spray rate was 3-12 g/min/kg, and the inlet temperature was 38-40° C. During the coating process, the Eudragit L-coated cores were maintained at 30-35° C. and the air volume was 130-160 m³/hr. A Eudragit S coating of approximately 10, 15, and 20% weight gain was coated onto the Eudragit L-coated cores to produce Formulations A, B, and C, respectively. The Eudragit S-coated multiparticulates were cooled in the Glatt GPCG-3 for 10 minutes post coating, then dried/cured in the following manner: 15 hours at 40° C.; cooled to 34° C. over 1 hour 45 min.; and then maintained at 34-35° C. for 7 hours 15 min. The multiparticulates were screened to remove oversized multiparticulates and fine material.

### Dissolution Testing of Formulations

The formulations were tested in dissolution test apparatuses to determine the rate at which drug was released from the formulations.

Gastro-Resistance Test Dissolution Media—pH 1.2 HCl: 172 mL of concentrated HCl was diluted to 20 L with de-ionized water. pH was adjusted to 1.15-1.25 by addition of concentrated HCl.

Extended release Test Dissolution Media—pH 6.8 Phosphate Buffer: 136.13 g KH₂PO₄ and 27.87 g NaOH was dissolved in 19 L of de-ionized water. Sodium hydroxide or phosphoric acid was added as necessary to adjust pH to 6.8 or 7.2 as appropriate.

Samples were first placed into 1000 mL of Gastro-Resistance Test Dissolution Media and tested in a USP Type I apparatus, at 100 rpm. After two hours of testing, baskets were carefully removed from the testing equipment and baskets were gently rinsed with approximately 15 mL of de-ionized water to remove traces of acid.

The contents of the baskets were promptly transferred to testing containers for a USP Type II apparatus in 1000 mL of Extended release Test Dissolution Media. Testing was performed at 100 rpm, with samples taken at 0.5, 1, 2, 3, 4, 6, 8, and 12 hours. Testing was performed at 37° C.

The in vitro drug release (% release by time) of the three base formulations is shown in Tables II and IIA below:

TABLE II

| | % Released | | |
|---|---|---|---|
| | Formulation A | Formulation B | Formulation C |
| | (omeprazole ER) | (omeprazole ER) | (omeprazole ER) |
| Time | 20 mg - | 20 mg - | 20 mg - |
| (hours) | “fast” release | “medium” release | “slow” release |
| 0.5 | 0.0 | 0.0 | 0 |
| 1.0 | 0.1 | 0.0 | 0 |
| 2.0 | 5.9 | 0.9 | 0 |
| 3.0 | 21.4 | 5.1 | 2 |
| 4.0 | 69.5 | 11.9 | 7 |
| 6.0 | 94.3 | 29.1 | 14 |
| 8.0 | 94.9 | 85.0 | 29 |
| 12.0 | 96.1 | 89.7 | 81 |

As can be seen from Table II, Formulation A released its contents the most quickly, followed by Formulation B, then Formulation C. Table IIA shows the results obtained when the samples were tested at pH 7.2.

TABLE IIA

| | % Released | | |
|---|---|---|---|
| | Formulation A | Formulation B | Formulation C |
| | (omeprazole ER) | (omeprazole ER) | (omeprazole ER) |
| Time | 20 mg - | 20 mg - | 20 mg - |
| (hours) | “fast” release | “medium” release | “slow” release |
| 0.5 | 0.0 | 0.0 | 0.0 |
| 1.0 | 0.3 | 0.0 | 0.0 |
| 2.0 | 14 | 1 | 0.4 |
| 3.0 | 56 | 11 | 4 |
| 4.0 | 95 | 21 | 12 |
| 6.0 | 95 | 87 | 23 |
| 8.0 | 95 | 92 | 70 |
| 12.0 | 95 | 91 | 88 |

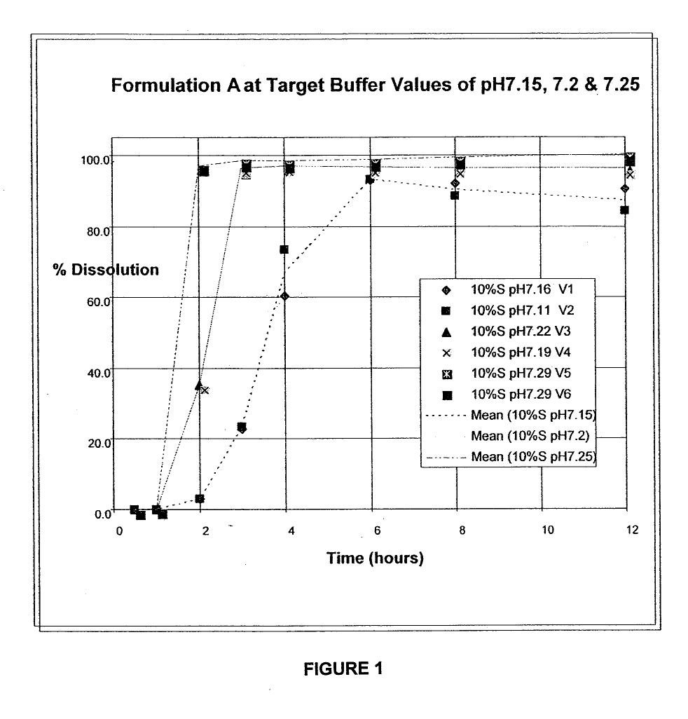

As can be seen from the differences between Tables II (pH 6.8) and IIA (pH 7.2), the formulations were highly sensitive to changes in pH around 7. To investigate further the pH dependence above pH 7, testing of Formulation A was at a series of different pH values, generally around 7.15, 7.20, and 7.25. Each pH was repeated. The results are presented in Table III below and are shown graphically in FIG. 1.

TABLE III

| | 10% S | 10% S | 10% S | 10% S | 10% S | 10% S | Mean | Mean | Mean |
|---|---|---|---|---|---|---|---|---|---|
| Time | pH 7.16 | pH 7.11 | pH 7.22 | pH 7.19 | pH 7.29 | pH 7.29 | (10% S | (10% S | (10% S |
| (hours) | V1 | V2 | V3 | V4 | V5 | V6 | pH 7.15) | pH 7.2) | pH 7.25) |
| 0 | | | | | | | 0 | 0 | 0 |
| 0.5 | 0.0 | 0.0 | 0.0 | 0.0 | 0.0 | 0.0 | 0 | 0 | 0 |
| 1 | 0.0 | 0.0 | 0.1 | 0.1 | 0.4 | 0.2 | 0 | 0 | 0 |
| 2 | 3.0 | 3.0 | 35.1 | 35.2 | 97.3 | 97.0 | 3 | 35 | 97 |
| 3 | 22.9 | 23.3 | 96.2 | 96.5 | 99.0 | 98.1 | 23 | 96 | 99 |
| 4 | 60.4 | 73.4 | 97.2 | 96.6 | 98.9 | 97.9 | 67 | 97 | 98 |
| 6 | 93.0 | 93.3 | 97.2 | 96.4 | 99.2 | 98.3 | 93 | 97 | 99 |
| 8 | 91.9 | 88.3 | 96.8 | 96.1 | 99.8 | 98.8 | 90 | 96 | 99 |
| 12 | 90.4 | 84.4 | 96.9 | 95.9 | 100.9 | 99.7 | 87 | 96 | 100 |

As can be seen from the results, very significant differences were observed between the different pH values. The percent released at 2 hours, for example, ranged from 3% at pH 7.15 to 97% at 7.25.

### Example 2

### In Vivo Performance of Formulations A, B, and C

While in vitro testing is a useful tool in preparing a pharmaceutical formulation, it is not always predictive of in vivo behavior. Indeed, when Formulations A, B, and C were tested in human subjects, as shown in this Example, surprising observations were made.

### Study Design

This biostudy was a multiple-dose, open label, randomized, crossover study. Sixteen healthy volunteer subjects were enrolled in this study. Subjects were dosed with a formulation of (extended release Formulations A, B, or C of omeprazole) once daily at night (approximately 22:00) for five days on three occasions or ^{Pr}LOSEC® (reference omeprazole product) twice daily in the evening before dinner (approximately 18:30), and in the morning before breakfast (approximately 08:00), for five days in a randomized crossover manner. Subjects were fasting for approximately three hours prior to and ten hours after the nighttime administration of the base Formulation A, B, or C. There was at least a seven-day washout period from the last dose in one period until the first dose of the subsequent period.

Blood samples (5 ml) were obtained prior to evening (^{Pr}LOSEC®) or nighttime (Formulations A, B, or C) dosing on Day 5, at 0.5, 1, 2, 3, 4, 6, and 8 hours after evening dosing with ^{Pr}LOSEC® on Day 5, prior to and at 0.5, 1, 2, 3, 4, 6, 8, and 10 hours after dosing with ^{Pr}LOSEC® the following morning and at 1, 2, 3, 4, 5, 6, 7, 8, 9, 10, 12, 14, 16, 20, and 24 hours after dosing with the extended release formulations on Day 5.

The gastric pH was recorded every four seconds over a 24 h period at baseline (Day—1) and on Day 5 using the MEDTRONICS digitrapper. The lower esophageal sphincter (LES) was located in the morning on Day—1, Period 1.

### Bioanalysis Methodology

Omeprazole was measured in plasma samples by a validated LC/MS/MS method incorporating a liquid/liquid extraction method by BioClin Research Laboratories. The limit of quantitation of the assay is 10 ng/ml and the assay range is 10-2500 ng/mL.

### Pharmacokinetics Methodology

The pharmacokinetic evaluation was conducted by PK Pharma Innovations Limited. The pharmacokinetic parameters were calculated using WinNonlin™, Version 4.0.1 (Pharsight Corporation, USA).

### Plasma

The following pharmacokinetic parameters were derived from the plasma concentrations versus time data for omeprazole, using non-compartmental methodology:
AUC₀₋ₜₐᵤ—Area under the curve from the time of dosing over the dosing interval calculated using the linear trapezoidal rule where AUC (t₁−t₂)=δt*(c₂+c₁)/2.
Maximum plasma concentration (Cₘₐₓ) and its corresponding time (tₘₐₓ) were recorded from the observed plasma concentration-time profiles. For the reference treatment, which was dosed bid, Cₘₐₓ and tₘₐₓ were recorded for each dosing interval.
Minimum plasma concentration (Cₘᵢₙ) and its corresponding time (tₘᵢₙ) were recorded from the observed plasma concentration-time profiles.
Average plasma concentration (C_{avg}) computed as AUC(₀₋ₜₐᵤ) divided by tau (dosing interval i.e. 24 hrs for the test treatments and 23.5 hrs for the reference treatment).
% fluctuation computed as 100*(Cₘₐₓ−Cₘᵢₙ)/C_{avg}, where Cₘᵢₙ and Cₘₐₓ were obtained between 0 and tau.
Relative bioavailability of the test treatments (Formulations A-C) to the reference (^{Pr}LOSEC®) (“D”) based on AUC (test/reference and expressed as a percentage).
T_{lag} is the time prior to the time corresponding to the first measurable (non-zero) concentration.

### Missing Samples and Spurious Data

Before a formal analysis, the pharmacokinetic data was subjected to a data review. This included checks for missing data and outliers. All the data was found to be in keeping with pharmacokinetic principles and pharmacokinetic analysis proceeded accordingly.

### Statistical and Graphical Methodology

The data was summarized using descriptive statistics. Arithmetic means, standard deviations, and coefficients of variation were calculated for the pharmacokinetics parameters listed. For each parameter, the median, minimum, and maximum values were presented. No formal statistical analysis was performed.

### PH Monitoring Methodology

### LES Identification

The location of the LES was determined manometrically. Prior to the pH probe insertion, the pH probes were calibrated at room temperature using the Medtronic buffer solution pH 1.07 and 7.01. The software used to process the pH data corrected for the difference between electrode calibration temperature (approx. 20° C.) and recording temperature (37° C.). The pH probe was inserted approximately 1 hour prior to start of the baseline pH on Day—1, Period 1 using the Medtronic Zinetics 24™ Single-Use pH Catheter with LES locator. The insertion of the pH probe could be facilitated by use of small amounts (e.g., 60 to 120 mL) of water as needed. On Day—1, Period 1 once the LES had been located the pH probe was positioned in the stomach 10 cm below the upper border of the LES. The baseline pH recording began at 08:00 on Day—1 of Period 1.

### PH Monitoring

Gastric pH was measured using the pH recording system (Digitrapper® 400, with a disposable pH probe (Medtronic Zinetics 24™ Single-Use pH Catheter with or without LES locator)). Prior to the pH probe insertion, the pH probes were calibrated at room temperature using the Medtronic buffer solution pH 1.07 and 7.01. The software used to process the pH data corrected for the difference between electrode calibration temperature (approx. 20° C.) and recording temperature (approx. 37° C.).

To measure gastric pH on Day—1 and 5 of each period, the pH probe was positioned in the stomach 10 cm below the upper border of the LES location determined on Day—1 of Period 1. The insertion of the pH probes could be facilitated by use of small amounts (e.g., 60 to 120 mL) of water as needed.

The pH probe was inserted approximately 1 hour prior to the start of the pH recording on Day—1 of each period for all subjects. On Day 5 of each period the pH probe was inserted at approximately 17:00 for all subjects.

The baseline pH recording began at 08:00 on Day—1 of each period. For subjects receiving ^{Pr}LOSEC® on Day 5 in each period, the pH recording began immediately after dosing at 18:00 (approximately one minute). For subjects receiving a test Formulation on Day 5 in each period, the pH recording began immediately after dosing at 22:00 (approximately one minute). The pH recording was performed for 24 hours. The 24-hour gastric pH measurements were conducted by trained staff.

### Analysis

The baseline and Day 5 median 24 h gastric pH, the time (h) and percentage time the gastric pH was less than 4, the integrated gastric pH (AUC, using the linear trapezoidal rule), and the occurrence of acid breakthrough in the 24 h period were calculated. In addition, the median gastric pH, time (h) and percentage time the gastric pH was less than 4, integrated gastric pH, and occurrence of acid breakthrough were calculated for the midnight to 3 am, 3 am to 6 am, 6 am to 9 am, 9 am to 12 noon, and 12 noon to 12 midnight timeframes. Acid breakthrough was assessed as intragastric pH <4 for a continuous period of at least an hour.

The change from baseline for all parameters, apart from the occurrence of acid breakthrough was analysed using analysis of variance (ANOVA). The occurrence of acid breakthrough was summarised as the number (percentage) of subjects with acid breakthrough.

Results

Pharmacokinetic

The plasma relative bioavailability (based on AUC₀₋ₜₐᵤ) of the test treatments compared to the reference product ranged from 76.7±28.0% (Formulation A—fast release), 63.3±27.5% (Formulation B—medium release), to 32.6±23.3% (Formulation C—slow release). The plasma Cₘₐₓ of the test treatments were 697.5±452.9 ng/mL (Formulation A—fast release), 514.5±372.6 ng/mL (Formulation B—medium release), and 236.4±258.6 ng/mL (Formulation C—slow release) compared to the reference product 759.1±269.6 ng/mL (first administration) and 782.0±358.3 ng/mL (second administration). The plasma C_{avg} of the test treatments were 128.6±91.2 ng/mL (Formulation A—fast release), 114.3±82.3 ng/mL (Formulation B—medium release), and 61.0±71.3 ng/mL (Formulation C—slow release) compared to the reference product 167.7±96.8 ng/mL. The plasma Cₘᵢₙ of the test treatments were 0.0±0.0 ng/mL (Formulation A—fast release), 1.7±6.7 ng/mL (Formulation B—medium release), and 0.0±0.0 ng/mL (Formulation C—slow release) compared to the reference product 4.0±15.9 ng/mL.

The percent fluctuation of the test treatments were 572.2±144.3% (Formulation A—fast release), 455.4±123.2% (Formulation B—medium release), 429.0±174.4% (Formulation C—slow release), compared to the reference product, which was 600.2±199.8%.

The lag times prior to the time corresponding to the first measurable (non-zero) concentrations were 3.6±1.3 h (Formulation A—fast release), 4.1±2.1 h (Formulation B—medium release), and 5.2±2.1 h (Formulation C—slow release) compared to the reference product, which was 1.6±2.3 h. The median time to reach peak plasma concentrations were 7 h (Formulation A—fast release), 8 h (Formulation B—medium release), and 9 h (Formulation C—slow release) compared to the reference product, 2 hr (first and second administration).

### PH Monitoring

Baseline

### Median pH

The baseline median pH ranged from 0.86±0.59 to 1.53±2.00 in the midnight to 3 am timeframe, from 1.32±1.58 to 1.82±1.94 in the 3 am to 6 am timeframe, from 1.85±1.58 to 2.39±1.92 in the 6 am to 9 am timeframe.

Time (% of time) pH <4

The time baseline pH was <4 ranged from 2.61±0.99 to 2.89±0.31 hours (86.90±32.88 to 96.46±10.29% of the time) in the midnight to 3 am timeframe, from 2.44±1.02 to 2.73±0.74 hours (81.30±34.09 to 91.02±24.72% of time) in the 3 am to 6 am timeframe, from 2.04±0.80 to 2.28±0.51 hours (67.97±26.71 to 75.86±16.84% of time) in the 6 am to 9 am timeframe.

### Integrated Gastric pH

The baseline integrated gastric pH (pH*min) ranged from 11,098±8,158 to 17,031±20,894 in the midnight to 3 am timeframe, from 15,615±16,619 to 21,744±21,070 in the 3 am to 6 am timeframe, from 25,414±10,728 to 31,501±15,568 in the 6 am to 9 am timeframe

Acid Breakthrough

Acid breakthrough occurred at baseline in 13-15 out of 16 (81.25-93.75%) subjects in the midnight to 3 am timeframe, 13-15 out of 16 subjects (81.25-93.75%) in the 3 am to 6 am timeframe, 9-12 out of 16 subjects (56.25-75.00%) in the 6 am to 9 am timeframe

Treatment A vs ^{Pr}LOSEC

:2-5 hours post dosing

The change from baseline in median pH, in time (% of time) that the pH was <4 and in integrated gastric pH in this timeframe was significantly less for Formulation A (Median pH: 0.30±0.41, p=0.0005; Time (% of time) pH <4: −0.15±0.28 hours (−4.85±9.25% of time), p=0.0008; Integrated gastric pH: 5,323±6,644, p=0.0002) than that for ^{Pr}LOSEC (Median pH: 2.29±1.79; Time (% of time) pH <4: −1.13±1.07 hours (−37.7±35.74% of time); Integrated gastric pH: 26,798±17,451). Six out of 16 subjects (37.50%) had acid breakthrough in this timeframe on ^{Pr}LOSEC compared to 9 out of 16 subjects (56.25%) who had acid breakthrough in this timeframe on Formulation A.

:5-8 hours post dosing

The change from baseline in median pH, in time (percent of time) that the pH was <4 and in integrated gastric pH in the 3 am to 6 am timeframe for Formulation A was not significantly different than that for ^{PR}LOSEC. Five out of 16 subjects (31.25%) on Formulation A and ^{Pr}LOSEC had acid breakthrough in the 3 am to 6 am timeframe.

:8-11 hours post dosing

The change from baseline in median pH, in time (% of time) the pH was <4 and in integrated gastric pH in the 6 am to 9 am timeframe was significantly more for Formulation A (Median ph: 3.71±1.91, p=0.0057; Time (% of time) pH <4: −1.70±0.84 hours (−56.7±28.16% of time), p=0.0012; Integrated gastric pH: 32,694±15,403, p=0.0011) than that for ^{Pr}LOSEC (Median pH: 2.36±1.79; Time (% of time) pH <4: −1.03±0.72 hours (−34.3±23.95% of time); Integrated gastric pH: 20,045±12,194). Two out of 16 subjects (12.50%) had acid breakthrough in the 6 am to 9 am timeframe on Formulation A and 4 out of 16 subjects (25.00%) had acid breakthrough in the 6 am to 9 am timeframe on ^{Pr}LOSEC.

### Overall

Formulation A performed significantly better than ^{Pr}LOSEC in the 8-11 hours post dosing period (6 am to 9 am timeframe)

Treatment B vs ^{Pr}LOSEC

:2-5 hours post dosing

The change from baseline in median pH, in time (% of time) pH was <4, and in integrated gastric pH in this timeframe was significantly less for Formulation B (Median pH: 0.99±1.82, p=0.0204: Time (% of time) pH <4: −0.45±0.81 hours (−14.8±27.15% of time), p=0.0152; Integrated gastric pH: 10,621±19,730, p=0.0037) than that for ^{Pr}LOSEC (Median pH: 2.29±1.79; Time (% of time) pH <4: −1.13±1.07 hours (−37.7±35.74% of time); Integrated gastric pH: 26,798±17,451). Six out of 16 subjects (37.50%) had acid breakthrough in this timeframe on ^{Pr}LOSEC compared to 9 out of 15 subjects (60.00%) who had acid breakthrough in this timeframe on Formulation B.

:3-8 hours post dosing

The change from baseline in median pH, in time (% of time) that the pH was <4 and in integrated gastric pH in the 3 am to 6 am timeframe for Formulation B was not significantly different than that for ^{PR}LOSEC. Five out of 16 subjects (31.25%) on ^{Pr}LOSEC had acid breakthrough in the 3 am to 6 am timeframe compared to 5 out of 15 subjects (33.33%) on Formulation B who had acid breakthrough in the 3 am to 6 am timeframe.

:8-11 hours post dosing

The change from baseline in median pH and in integrated gastric pH in the 6 am to 9 am timeframe was not significantly different from that for ^{Pr}LOSEC and Formulation B. The change from baseline in time (% of time) the pH was <4 in the 6 am to 9 am timeframe was significantly more for Formulation B (−1.47±0.95 hours (−49.1±31.61% of time), p=0.0358) compared to ^{Pr}LOSEC (−1.03±0.72 hours (−34.3±23.95% of time)). Two out of 15 subjects (13.33%) had acid breakthrough in the 6 am to 9 am timeframe on Formulation B and 4 out of 16 subjects (25.00%) had acid in this timeframe for Formulation B and ^{Pr}LOSEC. Five out of 16 subjects (31.25%) had acid breakthrough in this timeframe on ^{Pr}LOSEC compared to 6 out of 15 subjects (40.00%) who had acid breakthrough in this timeframe on Formulation B.

### Overall

Formulation B performed better than ^{Pr}LOSEC in the 8-11 hours post dosing period (6 am to 9 am timeframe).

### Treatment C vs ^{Pr}LOSEC

:2-5 hours post dosing

The change from baseline in median pH, in time (% of time) pH was <4 and in integrated gastric pH in this timeframe was significantly less for Formulation C (Median pH: −0.36±2.00, p<0.0001; Time (% of time pH <4: 0.21±0.93 hours (6.92±31.05% of time), p<0.0001; Integrated gastric pH: −1,236±19,732, p<0.0001) than that for ^{Pr}LOSEC (Median pH: 2.29±1.79; Time (% of time) pH <4: −1.13±1.07 hours (−37.7±35.74% of time); Integrated gastric pH: 26,798±17,451). Six out of 16 subjects (37.50%) had acid breakthrough in this timeframe on ^{Pr}LOSEC compared to 8 out of 16 subjects (50.00%) who had acid breakthrough in this timeframe on Formulation C.

:5-8 hours post dosing

The change from baseline in median pH, in time (% of time) that the pH was <4 and in integrated gastric pH in the 3 am to 6 am timeframe was significantly less for Formulation C (Median pH: 0.58±2.44, p=0.0009; Time (% of time) pH <4: −0.15±1.12 hours (−4.90±37.43% of time), p<0.0001; Integrated pH: 5,359±23,239, p<0.0001) than for ^{Pr}LOSEC (Median pH: 2.99±2.31; Time (% of time) pH <4: −1.37±1.01 hours (−45.8±33.50% of time); Integrated pH: 29,481±18,480). Five out of 16 subjects (31.25%) on ^{Pr}LOSEC had acid breakthrough in the 3 am to 6 am timeframe compared to 8 out of 16 subjects (50.00%) had acid breakthrough on Formulation C in this timeframe.

:8-11 hours post dosing

The change from baseline in median pH, in time (% of time) the pH was <4, and in integrated gastric pH in the 6 am to 9 am timeframe was not significantly different from that for ^{Pr}LOSEC for Formulation C. Two out of 16 subjects (12.50%) had acid breakthrough in the 6 am to 9 am timeframe on Formulation C and 4 out of 16 subjects (25.00%) had acid breakthrough in the 6 am to 9 am timeframe on ^{Pr}LOSEC.

### Overall

Formulation C performed similar to ^{PR}LOSEC in the 6 am to 9 am timeframe (8-11 hours post dosing).

### Clinical

All treatments were well tolerated in this healthy volunteer population.

### Discussion

The rank order which was observed in the in vitro dissolution of the test treatments (Formulation A fast release>Formulation B medium release>Formulation C slow release) was mirrored in a reduced relative bioavailabilities and % fluctuations (Formulation A fast release>Formulation B medium release>Formulation C slow release) compared to the reference treatment (Formulation D). In addition, the rank order which was observed in the in vitro dissolution of the test treatments (Formulation A fast release>Formulation B medium release>Formulation C slow release) was also mirrored in both the median time to reach peak plasma concentrations and the time corresponding to the first measurable (non-zero) plasma concentration (T_{lag}) for omeprazole.

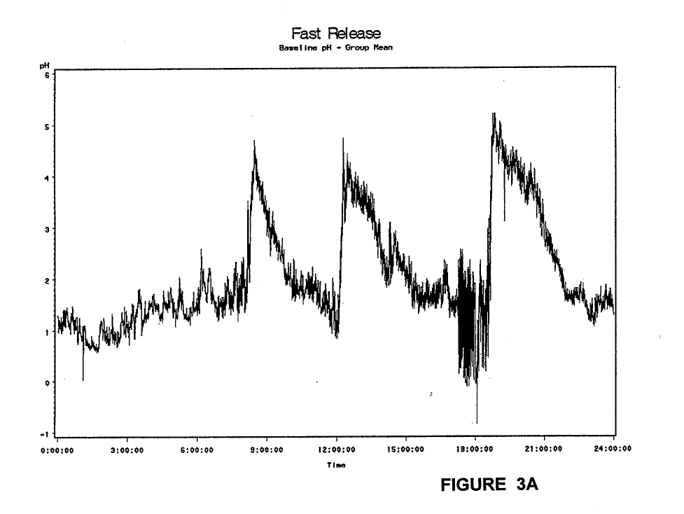

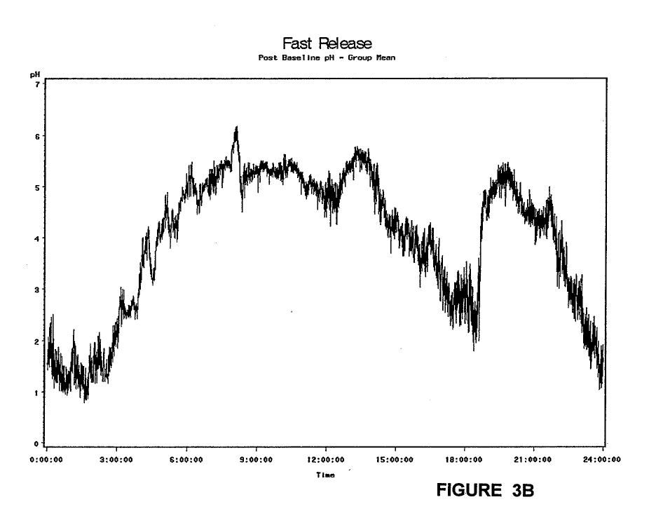

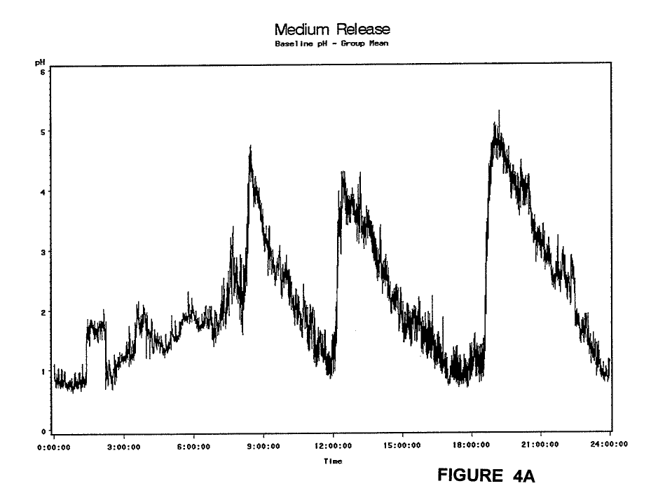

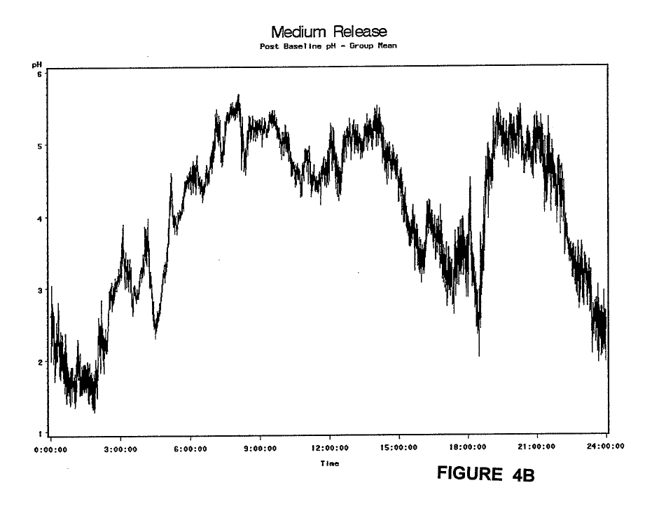

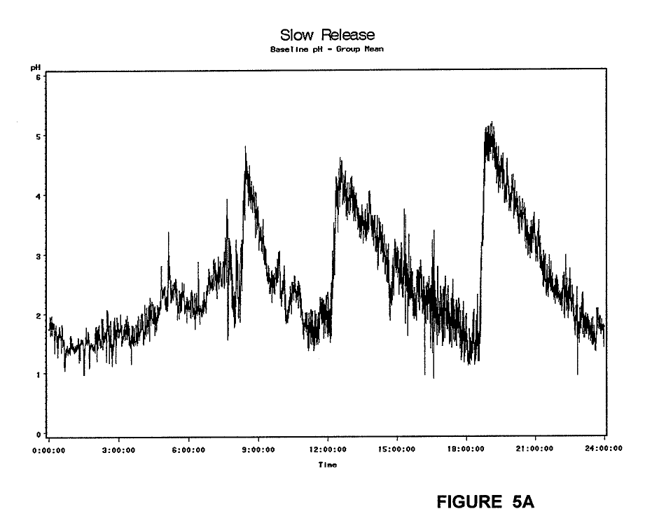

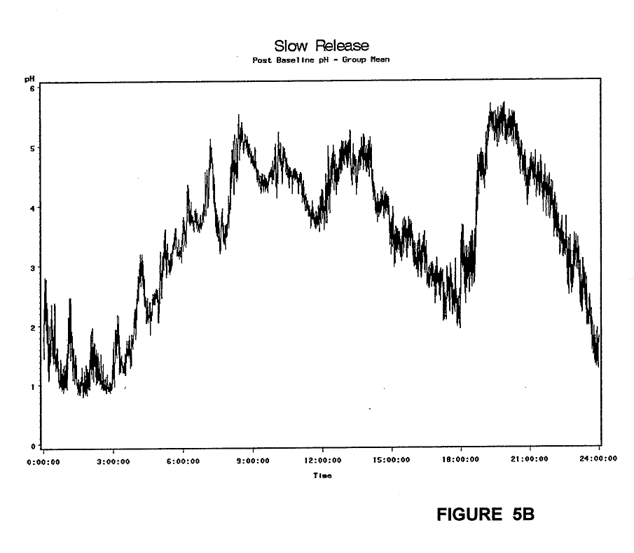

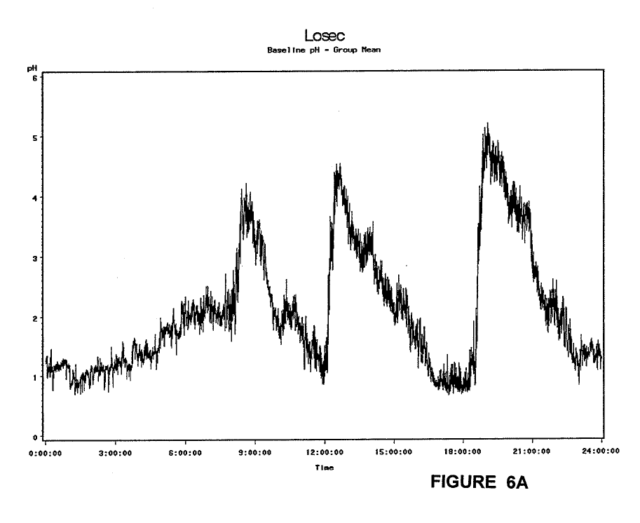

The aim of the test Formulations was to create “base” formulations that could be used individually or in combination with other omeprazole releasing components to improve the delivery profile for PPI drugs, which will significantly improve the pattern of acid control at night and reduce the incidence of NAB. The time of administration may be adapted to the observed time course of plasma omeprazole exposure and impact on gastric pH, and in particular, the period of peak nocturnal acid breakthrough, i.e., approximately from midnight to 6 am, and in particular in the peak NAB period of midnight to 3 am. FIGS. 3-6 show representative acid profiles for Formulations A, B, C, and the reference product, respectively. (In each Figure, the top frame is the normal pH profile and the bottom is the pH profile with treatment.)

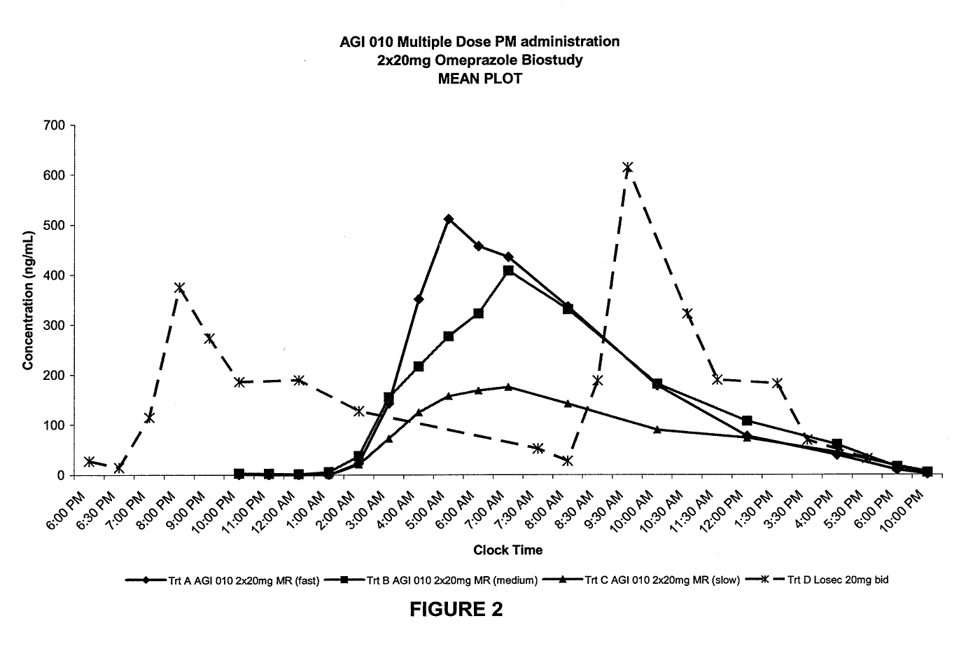

The mean plasma concentrations for the test and reference formulations are presented by clock times in FIG. 2. It is apparent from this graph that the release of omeprazole from the test Formulations was delayed until 3-4 hours post dosing (1-2 am), resulting in plasma concentration lower than ^{Pr}LOSEC in the midnight to 3 am timeframe. From 3 am-6 am, omeprazole was absorbed steadily from the three test Formulations, albeit at different rates depending on their in vitro release rates. Plasma levels were maintained at higher levels than ^{Pr}LOSEC in the 3 am to 9 am timeframe, (5-11 hours post dosing) resulting in median time to peak concentrations between 5 am (7 hours post dosing) (fast release—Formulation A) and 7 am (9 hours post dosing) (slow release—Formulation C) depending on the release properties of the test Formulation. In comparison, the levels of omeprazole from the reference treatment (^{Pr}LOSEC b.i.d.) were declining over the same period, reaching a nadir at 8 am.

In terms of gastric acid control for the 3 am to 6 am (5-8 hours post dosing) timeframe, Formulations A and B performed similarly to ^{Pr}LOSEC, whereas Treatment C performed significantly poorer to ^{Pr}LOSEC in this timeframe. Treatment A performed significantly better than ^{Pr}LOSEC in the 6 am to 9 am timeframe (8-11 hours post dosing), Treatment B also performed better than ^{Pr}LOSEC in this timeframe and Treatment C performed similarly to ^{Pr}LOSEC in this timeframe.

### Conclusions

The reference product, ^{Pr}LOSEC, includes an enteric coating to prevent its release in the stomach, but beyond that, has no additional features that result in a delay in drug release. As each of the test formulations included an additional coating—over an enteric coating—which resulted in an additional delay in drug release, none of the test Formulations resulted in a rapid drug level in the blood and thus, following dosing at 10 pm at night, none controlled acid in the early midnight to 3 am period of the night.

During the 3-6 am time frame, the faster releasing Formulations, A and B, performed similarly to the reference product. However, during the 6-9 am period (8-11 hours post dosing), the slowest releasing Formulation, C, performed as well as the reference product, whereas Formulations B and A performed slightly better and significantly better. The reason for this increase in performance can be seen in FIG. 2, which shows that the plasma drug level for Formulation A was the highest during this period, followed by Formulation B, then C, and finally the reference formulation.

Based on the pharmacokinetic and pH data it is clear that by administering the Formulations A or B at an earlier time (e.g., around dinnertime (6 pm)), then the subsequent pattern of omeprazole exposure and associated intragastric pH can be aligned with the period of NAB.

The pattern of intragastric pH control also suggested that incorporation of an earlier releasing component in combination with the more delayed/extended release Formulation A or B components would further optimize the control of intragastric pH during the NAB period.

The unexpected loss in bioavailability progressively from Formulation A to B to C both explains the lack of adequate pH control with Formulation C and suggests a further benefit to be gained in intragastric pH control by improving the bioavailability of Formulation A and particularly Formulation B.

A further interesting discovery was made when comparing test Formulations A, B, and C. Each test Formulation included a coating, which should have delayed the release in vivo, as predicted by the increased delays shown in vitro. However, the Tₘₐₓ was not as time-shifted as might be expected from the in-vitro profiles of Formulation A to B to C, but rather was decreased from Formulation A to B to C. This result suggests that the window for PPI bioavailability from the GI tract may be narrower than has previously been believed. It further suggests that formulations that are designed to release a PPI into the GI tract for a period of 12 hours or longer may not result in drug bioavailability in the latter hours of release.

Based upon these studies, it is clear that Formulations A and B can be used as an extended release component alone, or as a “second” component in a mixed formulation, combined with a more fast-releasing (i.e., a delayed-release, or “first”) component.

Table IV summarizes the pharmacokinetic parameters observed from these studies.

TABLE IV

| | Formulation A | Formulation B | Formulation C | Losec |
|---|---|---|---|---|
| | (omeprazole MR) | (omeprazole MR) | (omeprazole MR) | 20 mg |
| PK Parameters | 20 mg - | 20 mg - | 20 mg - | BID |
| (Mean ± SD - CV %) | fast release | medium release | slow release | administration |
| Relative | 76.403 ± 27.795 | 62.747 ± 27.744 | 32.337 ± 23.312 | — |
| Bioavailability (%) | | | | |
| CV % | 36.4 | 44.2 | 72.1 | |
| (based on AUCₗₐₛₜ) | | | | |
| AUCₗₐₛₜ (ng/mL · h) | 3055.195 ± 2180.865 | 2708.775 ± 1974.145 | 1447.420 ± 1706.711 | 3925.475 ± 2290.986 |
| CV % | 71.4 | 72.9 | 117.9 | 58.4 |
| Cmax (ng/mL) | 697.547 ± 452.881 | 514.458 ± 372.578 | 236.434 ± 258.631 | 872.866 ± 287.741 |
| CV % | 64.9 | 72.4 | 109.4 | 33.0 |
| Cmin (ng/mL) | 0.000 ± 0.000 | 1.720 ± 6.662 | 0.000 ± 0.000 | 3.982 ± 15.928 |
| CV % | . | 387.3 | . | 400.0 |
| Cavg (ng/mL) | 128.649 ± 91.157 | 114.315 ± 82.315 | 61.012 ± 71.280 | 164.394 ± 98.510 |
| CV % | 70.9 | 72.0 | 116.8 | 59.9 |
| % Fluctuation (%) | 572.156 ± 144.264 | 455.412 ± 123.200 | 429.010 ± 174.368 | 624.235 ± 205.766 |
| CV % | 25.2 | 27.1 | 40.6 | 33.0 |
| Tmax (h) | 7.813 ± 1.721 | 7.600 ± 1.639 | 8.625 ± 2.062 | 10.094 ± 6.293 |
| CV % | 22.0 | 21.6 | 23.9 | 62.3 |
| Median | 7.00 | 8.00 | 9.00 | 14.50 |
| Range | 6.00-12.00 | 5.00-10.00 | 5.00-12.00 | 1.00-15.50 |
| Tmin (h) | 0.063 ± 0.250 | 0.267 ± 0.704 | 0.063 ± 0.250 | 1.219 ± 3.488 |
| CV % | 400.0 | 263.9 | 400.0 | 286.2 |
| Median | 0.00 | 0.00 | 0.00 | 0.00 |
| Range | 0.00-1.00 | 0.00-2.00 | 0.00-1.00 | 0.00-14.00 |
| Tlag (h) | 3.563 ± 1.315 | 4.067 ± 2.086 | 5.188 ± 2.105 | 1.563 ± 2.330 |
| CV % | 36.9 | 51.3 | 40.6 | 149.1 |

### Example 3

### Dosing at Dinnertime

This Example presents a multiple-dose, open label, randomized, crossover study evaluating the steady state pharmacokinetics and effect on gastric pH of test formulations dosed before or after dinner for five days in healthy volunteers compared to Prilosec®.

Study population: Sixteen (16) normal healthy volunteer subjects were enrolled in this study to ensure completion of 12 subjects. Fifteen subjects completed all four treatment periods.

Test Formulations and Treatments

The test formulations used in this Example make use of: 1) Eudragit L-coated cores coated to a 10% weight gain with a Eudragit S solution (described as Formulation A in Example 1); and 2) Eudragit L-coated cores coated to a 7.5% weight gain with a Eudragit S solution (prepared as described in Example 1 above, except to a 7.5% weight gain).

Treatments: The treatments were as follows:

Treatment A—40 mg of omeprazole in the form of two 20-mg capsules of 10% S polymer coated beads (Formulation A from Example 1) in a capsule administered 30 minutes before dinner.

Treatment B—40 mg of omeprazole in the form of two 20-mg capsules of 7.5% S polymer coated beads in a capsule administered 30 minutes before dinner.

Treatment C—40 mg of omeprazole in the form of two 20 mg capsules of 7.5% S polymer coated beads in a capsule administered 30 minutes after dinner.

Treatment D—40 mg of Prilosec® (40 mg capsules of the FDA approved prescription Prilosec®) administered 30 minutes before dinner.

Results

All test treatments showed delays in the onset of absorption with delayed median tₘₐₓ values ranging from 5 to 9 hours and extended lag times (t_{lag}) compared with the reference product Prilosec.

The results of the effects of the various treatments on intragastric pH during the 3 am to 6 am period are presented below in Tables V-IX.

TABLE V
Time Gastric pH < 4 for 3 am-6 am Time Interval.

| | | B. 40 mg; | C. 40 mg; | D. 40 mg; | E. |
|---|---|---|---|---|---|
| | A. | 10% S | 7.5% S Pre- | 7.5% S | Prilosec |
| | Baseline | Pre-dinner | dinner | Post-dinner | 40 mg |
| N | 16 | 16 | 15 | 16 | 16 |
| 3 am-6 am | | | | | |
| Mean | 95.7 | 54.6 | 55.0 | 49.0 | 62.3 |
| SD | 13.0 | 32.8 | 36.6 | 39.0 | 39.6 |

The baseline value for a given subject was the mean of the four individual baseline determinations. Values are the percent of time pH < 4 from the number of subjects and the treatment indicated.

TABLE VI
Median Gastric pH for 3-6 h Time Intervals.

| | | | | D. 40 mg; | |
|---|---|---|---|---|---|
| | | B. 40 mg; | C. 40 mg; | 7.5% S | E. |
| | A. | 10% S | 7.5% S | Post- | Prilosec |
| | Baseline | Pre-dinner | Pre-dinner | dinner | 40 mg |
| N | 16 | 16 | 15 | 16 | 16 |
| 3 am-6 am | | | | | |
| Median | 1.19 | 4.24 | 3.44 | 4.46 | 2.80 |
| Interquartile | 1.13-1.20 | 1.87-4.89 | 2.05-5.53 | 2.75-5.63 | 1.60-5.27 |
| Range | | | | | |

The baseline value for a given subject was the median of the four individual baseline determinations. Values are medians from the number of subjects and the treatment indicated.

TABLE VII
Integrated Gastric Acidity for 3 am-6 am Time interval.

| | | B. 40 mg; | C. 40 mg; | D. 40 mg; | E. |
|---|---|---|---|---|---|
| | A. | 10% S | 7.5% S Pre- | 7.5% S | Prilosec |
| | Baseline | Pre-dinner | dinner | Post-dinner | 40 mg |
| N | 16 | 16 | 15 | 16 | 16 |
| 3 am-6 am | | | | | |
| Mean | 207.6 | 39.8 | 32.6 | 29.0 | 49.5 |
| SD | 44.2 | 55.9 | 39.8 | 49.7 | 53.3 |

The baseline value for a given subject was the mean of the four individual baseline determinations. Values are integrated gastric acidity (mmol · hr/L) from the number of subjects and the treatment indicated.

TABLE VIII
Inhibition Integrated Gastric Acidity for 3am-6am Time Interval.

| | B. 40 mg; | C. 40 mg; | | |
|---|---|---|---|---|
| | 10% | 7.5% | D. 40 mg; 7.5% | E. Prilosec |
| | S Pre-dinner | S Pre-dinner | S Post-dinner | 40 mg |
| N | 16 | 15 | 16 | 16 |
| 3 am-6 am | | | | |
| Mean | 81.4 | 85.0 | 86.1 | 76.4 |
| SD | 26.5 | 18.9 | 23.6 | 25.0 |

The baseline value for a given subject was the mean of the four individual baseline determinations. Inhibition was calculated as 100 × (Baseline − Value)/Baseline. Values are for inhibition (%) from the number of subjects and the treatment indicated.

TABLE IX
Acid Breakthrough for 3 am-6 am Time Interval.

| | | B. 40 mg; | C. 40 mg; | D. 40 mg; | E. |
|---|---|---|---|---|---|
| | A. | 10% S | 7.5% S Pre- | 7.5% S | Prilosec |
| | Baseline | Pre-dinner | dinner | Post-dinner | 40 mg |
| Total | 16 | 16 | 15 | 16 | 16 |
| 3 am-6 am | 16 | 6 | 9 | 3 | 9 |
| Percent | 100 | 38 | 60 | 19 | 56 |

The baseline value for a given subject was calculated as the mean number of minutes gastric pH < 4 for the four individual baseline determinations. Values are for the number of subjects with gastric pH < 4 for 60 consecutive minutes with the treatment indicated.

The results of this Example show that after dosing the test formulations at 6 pm, around dinnertime, there is an improved control of intragastric pH in the NAB period of 3 am to 6 am. This confirms the particular utility of these single-component formulations when dosed at dinnertime to improve intragastric pH control during the latter segment of the NAB period. However to further improve control of NAB, it was determined to assess the impact of 2-component formulations on the profile of intragastric pH control during the peak NAB period of midnight to 3 am (See Example 4 below).

### Example 4

### Combination Formulations Dosed at Dinnertime

This biostudy was a multiple-dose, open label, randomized, crossover study. Sixteen healthy volunteer subjects were enrolled in this study. Subjects were dosed with one of three formulations (extended release formulation of omeprazole) once daily at night 60 minutes before dinner on three different occasions or with PRILOSEC® once daily at night 30 minutes before dinner for five days in a randomized crossover manner. All doses were administered with 240 ml of water. There was at least a seven-day washout period from the last dose in one period until the first dose of the subsequent period.

The formulations used in this example make use of: 1) Eudragit L-coated cores (which are described as “Eudragit L-coated cores” in Example 1 above); 2) Eudragit L-coated cores coated to a 10% weight gain with a Eudragit S solution (described as Formulation A in Example 1); and 3) Eudragit L-coated cores coated to a 15% weight gain with a Eudragit S solution (described as Formulation B in Example 1).

The formulations used within this Example were: 1) Treatment 1—Prilosec® 40 mg (40 mg capsules of the FDA approved prescription Prilosec®) administered 30 minutes before dinner; 2) Treatment 2—40 mg of omeprazole in the form of one 10-mg capsule of Eudragit L-coated cores and two 15-mg capsules of 15% Eudragit S-coated beads (Formulation B) administered 60 minutes before dinner (“10/30/15%”); 3) Treatment 3—40 mg of omeprazole in the form of two 10-mg capsules of Eudragit L-coated beads and one 20-mg capsule of 15% Eudragit S-coated beads (Formulation B) administered 60 minutes before dinner (20/20/15%); and Treatment 4—40 mg of omeprazole in the form of one 10 mg capsule of Eudragit L-coated beads and two 15-mg capsules of 10% Eudragit S-coated beads (Formulation A) administered 60 minutes before dinner (10/30/10%).

These formulations were selected in order maximise the intragastric pH control during the peak NAB period of midnight to 3 am following administration at dinner time.

Plasma and pH Sampling—Blood samples (5-ml) were obtained prior to evening dosing on Day 5, at 0.5, 1, 2, 3, 4, 5, 6, 7, 8, 9, 10, 12, 16, 20, and 24 hours. The gastric pH was recorded every four seconds over a 24 h period at baseline (Day—1) and on Day 5 using a MEDTRONICS digitrapper. The lower esophageal sphincter (LES) was located in the morning on Day—1, Period 1.

Pharmacokinetics Methodology—The bioanalytical methods, pharmacokinetic methods, and the corresponding results are presented briefly below:

Bioanalysis Methodology—Omeprazole was measured in plasma samples by a validated LC/MS/MS method incorporating a liquid/liquid extraction method by BioClin Research Laboratories. The limit of quantitation of the assay is 10 ng/ml and the assay range is 10-2500 ng/mL.

Pharmacokinetic Analysis—The pharmacokinetic evaluation were calculated using WinNonlin™, Version 4.0.1 (Pharsight Corporation, USA).

The following pharmacokinetic parameters were derived from the plasma concentrations versus time data for omeprazole, using non-compartmental methodology:

AUC0-tau—Area under the curve from the time of dosing over the dosing interval calculated using the linear trapezoidal rule where AUC (t1−t2)=δt*(c2+c1)/2.

Maximum plasma concentration (Cmax) and its corresponding time (tₘₐₓ) were recorded from the observed plasma concentration-time profiles. For the reference treatment, which was dosed bid, Cmax and tₘₐₓ were recorded for each dosing interval.

Relative bioavailability of the test treatments (treatments 2-4) to the reference (treatment 1) based on AUC (test/reference and expressed as a percentage).

T_{lag} is the time prior to the time corresponding to the first measurable (non-zero) concentration

Missing Samples and Spurious Data—Before a formal analysis, the pharmacokinetic data was subjected to a data review. This included checks for missing data and outliers. All the data was found to be in keeping with pharmacokinetic principles and pharmacokinetic analysis proceeded accordingly.

Statistical and Graphical Methodology—The data was summarized using descriptive statistics. Arithmetic means, standard deviations, and coefficients of variation were calculated for the pharmacokinetics parameters listed. For each parameter, the median, minimum, and maximum values were presented. No formal statistical analysis was performed. The mean, treatment, and individual subject concentrations versus time profiles were also prepared. All graphs were prepared using WinNonlin.

### pH Monitoring Methodology

The location of the LES was determined manometrically. Prior to the pH probe insertion, the pH probes were calibrated at room temperature using the Medtronic buffer solution pH 1.07 and 7.01. The software used to process the pH data corrected for the difference between electrode calibration temperature (approx. 20° C.) and recording temperature (37° C.). The pH probe was inserted approximately 1 hour prior to start of the baseline pH on Day—1, Period 1 using the Sandhill pH Catheter with LES locator. The Zinetics Catheter used in study AGI010-001 was not available for use in this study so Medtronic instructed the site to use the Sandhill Catheter. The insertion of the pH probe could be facilitated by use of small amounts (e.g., 60 to 120 mL) of water as needed. On Day—1, Period 1 once the LES had been located the pH probe was positioned in the stomach 10 cm below the upper border of the LES. The baseline pH recording began at 08:00 on Day—1 of Period 1.

Gastric pH was measured using the pH recording system (Digitrapper® 400) with a disposable pH probe (Sandhill pH Catheter with or without LES locator). Prior to the pH probe insertion, the pH probes were calibrated at room temperature using the Medtronic buffer solution pH 1.07 and 7.01.

To measure gastric pH on Day—1 and 5 of each period, the pH probe was positioned in the stomach 10 cm below the upper border of the LES location determined on Day—1 of Period 1. The insertion of the pH probes could be facilitated by use of small amounts (e.g., 60 to 120 mL) of water as needed.

The pH probe was inserted approximately 1 hour prior to the start of the pH recording on Day—1 of each period for all subjects. On Day 5 of each period the pH probe was inserted at approximately 17:00 for all subjects.

The baseline pH recording began at 08:00 on Day—1 of each period. For subjects receiving Prilosec® on Day 5 in each period the pH recording began immediately after dosing at 18:00 (approximately one minute). For subjects receiving test formulations on Day 5 in each period the pH recording began immediately after dosing at 22:00 (approximately one minute). The pH recording was performed for 24 hours. The 24-hour gastric pH measurements were conducted by trained staff.

The pH data was initially downloaded from the Medtronic portable data storage unit and placed directly into SAS for analysis without any correction factor for the difference between electrode calibration temperature (approx. 20° C.) and recording temperature (approx. 37° C.).

To correctly analyze the pH, data were transferred electronically from the Medtronic portable data storage unit and processed using software designed for pH recordings (Polygram98, Medtronic Synectics, Shoreview, Minn.). When data are exported in ASCII format using Polygram98 software, the program takes the individual pH values that are recorded every 4 seconds and fills in the same value for the other seconds resulting in one value for every second of the recording period. These pH data were rescaled using the new Medtronic temperature correction factors as described previously. The rescaled baseline values were used for all calculations. For the present report, median pH, time gastric pH <4 (expressed as a percentage of 24 hours), integrated gastric acidity, percent inhibition of integrated gastric acidity, and incidence of NAB were calculated for the 24-hour period. In addition, the pH metrics were also calculated for the midnight to 3 am, 3 am to 6 am, 6 am to 9 am, and 9 am to 12 noon. Acid breakthrough was assessed as intragastric pH <4 for a continuous period of at least an hour. The occurrence of acid breakthrough was summarised as the number and percentage of subjects with acid breakthrough.

### Pharmacokinetic and Pharmacodynamic Results

A total of 16 subjects (6 females and 10 males) were randomized to receive single oral doses of omeprazole (Treatments 1-4) for five days in each of 4 periods.

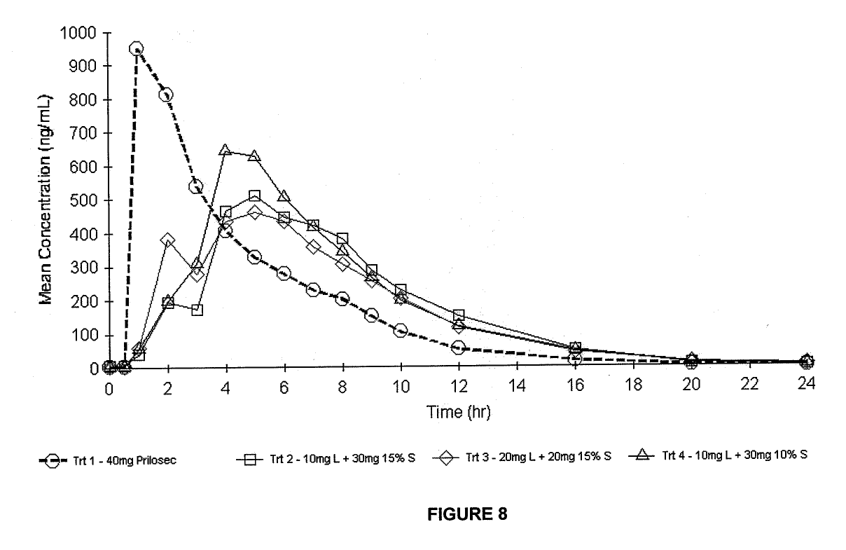

Briefly, Tables XI and XII provide a summary of the pharmacokinetic parameters for the 4 treatments, with FIG. 8 representing the mean plots of the treatments. The results show that the actual plasma profiles of the tested formulations were very similar to the desired, predicted profiles with an AUC more similar to the reference compared to Example 1, a Cₘₐₓ more similar to the reference, a decrease in lag time, a decrease in Tₘₐₓ, and a two-peak profile with the earlier initial peak having a lower maximum concentration than the later peak. The mean relative bioavailability (based on AUC₀₋ₜₐᵤ of the plasma concentration) of the test treatments compared to the reference product were 102.77%-109.46%% for the three test formulations of this example. The mean Cₘₐₓ ratios were 93.22%-105.32%.

The mean lag times (T_{lag}) for the tested formulations were longer than the reference product—0.91 hr for 10/30/15%, 0.85 hr for 20/20/15%, 0.75 hr for 10/30/10%, and 0.44 hr for Prilosec®. The median time to reach peak plasma concentrations (Tmax) were 5 hr (10/30/15%), 5 hr (20/20/15%), 4 hr (10/30/10%), and 1.0 hr (Prilosec®).

TABLE X
Mean Omeprazole pharmacokinetic parameters by Treatment

| PK | | | Treatment 3 - | Treatment 4 - |
|---|---|---|---|---|
| Parameters | Treatment 1 - | Treatment 2 - | 20 mg L + 20 mg | 10 mg L + 30 mg |
| (Mean ± SD - | 40 mg Prilosec | 10 mg L + 30 mg 15% S | 15% S | 10% S |
| CV %) | N = 16 | N = 16 | N = 16 | N = 16 |
| AUC₀₋ₜₐᵤ | 4002.571 ± 3209.022 | 3933.054 ± 3081.128 | 3848.577 ± 2280.129 | 4254.517 ± 4113.656 |
| (ng/mL · hr) | | | | |
| CV % | 80.2 | 78.3 | 59.2 | 96.7 |
| AUC_{0-inf} | 4048.976 ± 3216.245 | 3988.532 ± 3094.997 | 3916.910 ± 2357.149 | 4300.983 ± 4148.893 |
| (ng/mL · hr) | | | | |
| CV % | 79.4 | 77.6 | 60.2 | 96.5 |
| Cₘₐₓ (ng/mL) | 1147.871 ± 765.363 | 718.809 ± 483.906 | 620.926 ± 306.113 | 848.067 ± 617.310 |
| CV % | 66.7 | 67.3 | 49.3 | 72.8 |
| Tₘₐₓ (hr) | 1.813 ± 1.974 | 5.625 ± 2.306 | 4.875 ± 2.391 | 4.625 ± 1.586 |
| CV % | 108.9 | 41.0 | 49.0 | 34.3 |
| Median | 1.00 | 5.00 | 5.00 | 4.00 |
| Range | 1.00-9.00 | 1.00-10.00 | 1.00-9.00 | 2.00-8.00 |
| T_{lag} (hr) | 0.438 ± 0.171 | 0.906 ± 0.584 | 0.844 ± 0.437 | 0.750 ± 0.483 |
| CV % | 39.0 | 64.4 | 51.7 | 64.4 |
| T_{half} (hr) | 1.758 ± 1.146 | 2.835 ± 2.906 | 2.386 ± 1.154 | 2.185 ± 0.981 |
| CV % | 65.2 | 102.5 | 48.4 | 44.9 |
| Duration of | 9.181 ± 3.484 | 10.266 ± 4.737 | 10.926 ± 4.784 | 9.923 ± 4.881 |
| time cover | 37.9 | 46.1 | 43.8 | 49.2 |
| plasma | | | | |
| concentration | | | | |
| >80 ng/mL (hr) | | | | |
| Duration of | 8.202 ± 3.419 | 9.488 ± 4.581 | 9.994 ± 4.437 | 8.980 ± 4.638 |
| time cover | 41.7 | 48.3 | 44.4 | 51.6 |
| plasma | | | | |
| concentration | | | | |
| >100 ng/mL (hr) | | | | |

TABLE XI
Mean Omeprazole pharmacokinetic comparisons

| PK Comparisons | | | Treatment 3 - | Treatment 4 - |
|---|---|---|---|---|
| Relative to Treatment 1 - | | Treatment 2 - | 20 mg L + 20 mg | 10 mg L + 30 mg |
| 40 mg Prilosec | | 10 mg L + 30 mg 15% S | 15% S | 10% S |
| (Mean ± SD - CV %) | | N = 16 | N = 16 | N = 16 |
| Relative | Based on | 102.77 ± 59.06 | 109.46 ± 68.17 | 104.58 ± 49.15 |
| Bioavailability | AUCₜₐᵤ | | | |
| (%) CV % | | 57.47 | 62.27 | 47.00 |
| (Trt1) | | | | |
| Relative | Based on | 100.23 ± 43.11 | 105.83 ± 50.72 | 102.83 ± 44.19 |
| Bioavailability | AUC_{inf} | | | |
| (%) CV % | | 43.02 | 47.92 | 42.98 |
| (Trt1) | | | | |

| | | | |
|---|---|---|---|
| Ratio Cmax | 100.49 ± 122.97 | 93.22 ± 101.96 | 105.32 ± 83.43 |
| (%) | 122.37 | 109.37 | 79.22 |

The median pH for the baseline and all administered dosage forms is presented in Table XII.

TABLE XII
Median Gastric pH for Midnight to 3 am.

| | | B. | C. | D. | E. |
|---|---|---|---|---|---|
| | A. | Prilosec | 0/30/15% | 20/20/15% | 10/30/10% |
| | Baseline | 40 mg | 40 mg | 40 mg | 40 mg |
| N | 16 | 16 | 16 | 16 | 16 |
| 0-3 am | | | | | |
| Median | 1.08 | 3.89 | 4.23 | 3.96 | 4.23 |
| Interquartile | 1.02-1.13 | 2.86-4.62 | 3.37-4.74 | 3.51-4.26 | 3.60-4.82 |
| Range | | | | | |

The baseline value for a given subject was the median of the four individual baseline determinations. Values are medians from the number of subjects and the treatment indicated.

The mean percent time pH <4 is presented in Table XIII.

TABLE XIII
Time Gastric pH < 4 for Midnight to 3 am.

| | | | | D. | E. |
|---|---|---|---|---|---|
| | A. | B. Prilosec | C. 0/30/15% | 20/20/15% | 10/30/10% |
| | Baseline | 40 mg | 40 mg | 40 mg | 40 mg |
| N | 16 | 16 | 16 | 16 | 16 |
| 0-3 am | | | | | |
| Mean | 100.0 | 48.9 | 49.3 | 52.0 | 40.7 |
| SD | 0.0 | 27.3 | 30.9 | 26.1 | 23.9 |

The baseline value for a given subject was the mean of the four individual baseline determinations. Values are time pH < 4 (%) from the number of subjects and the treatment indicated.

The gastric acidity results are presented in Tables XIV-XVI.

TABLE XIV
Integrated Gastric Acidity for Midnight to 3 am

| | | | | D. | E. |
|---|---|---|---|---|---|
| | A. | B. Prilosec | C. 0/30/15% | 20/20/15% | 10/30/10% |
| | Baseline | 40 mg | 40 mg | 40 mg | 40 mg |
| N | 16 | 16 | 16 | 16 | 16 |
| 0-3 am | | | | | |
| Mean | 264.2 | 18.9 | 16.8 | 9.8 | 8.2 |
| SD | 47.1 | 34.1 | 34.2 | 11.0 | 13.5 |

The baseline value for a given subject was the mean of the four individual baseline determinations. Values are integrated gastric acidity (mmol · hr/L) from the number of subjects and the treatment indicated.

TABLE XV
Inhibition Integrated Gastric Acidity for Midnight to 3am

| | B. Prilosec | C. 0/30/15% | D. 20/20/15% | E. 10/30/10% |
|---|---|---|---|---|
| | 40 mg | 40 mg | 40 mg | 40 mg |
| N | 16 | 16 | 16 | 16 |
| 0-3 am | | | | |
| Mean | 92.1 | 93.4 | 96.4 | 97.1 |
| SD | 16.3 | 13.9 | 4.0 | 4.3 |

TABLE XVI
Acid Breakthrough for Midnight to 3 am

| | | | C. | D. | E. |
|---|---|---|---|---|---|
| | | B. Prilosec | 0/30/15% | 20/20/15% | 10/30/10% |
| | A. Baseline | 40 mg | 40 mg | 40 mg | 40 mg |
| Total | 16 | 16 | 16 | 16 | 16 |
| 0-3 am | 16 | 4 | 2 | 2 | 2 |
| Percent | 100 | 25 | 13 | 13 | 13 |

The baseline value for a given subject was calculated as the mean number of minutes gastric pH < 4 for the four individual baseline determinations. Values are for the number of subjects with gastric pH < 4 for 60 consecutive minutes with the treatment indicated. Percent refers to the total number of subjects.

### Discussion

Based on the results with 10 pm dosing of Formulations A and B in Example 2 and the results from 6 pm dosing in Example 3, it was apparent that earlier dosing with these formulations—around 6 pm—should better align the impact of omeprazole on intragastric pH to the NAB period and in particular the peak NAB period of midnight to 3 am. This Example, together with Example 3 above, demonstrate that formulations according to the invention can be tailored to provide control of stomach pH during specific periods during the night, depending on the needs of the user. Thus, NAB can be treated at any period during sleeping hours, using the present invention.

The test formulations in this current example were designed to further improve the delivery profile of omeprazole by combining either the Formulation A or B extended release components with earlier-releasing delayed release components (referred to as L-coated pellets or beads).

It was apparent from the mean plasma concentration curves that the release of omeprazole from the test formulations was typically extended and peak plasma concentrations occurred sometime between 8 pm to 2 am. Given the design of the test formulations and the difference in their in vivo release compared to Prilosec®, the plasma concentrations for the test formulations were predictably lower than Prilosec® from 6 pm to approximately 10 pm but greater than Prilosec® from 10 pm to 12 pm the next day.

It was surprisingly found that by incorporating earlier-releasing components in combination with the Formulation A and Formulation B components that the bioavailability was improved. Without being bound by any specific theory, it is believed that the earlier releasing component saturates a metabolism of omeprazole so that the slower releasing component is not as extensively metabolized.

During the morning hours (midnight to 9 am), trends were found when comparing the plasma concentration and pH profiles.

1. The plasma concentration and the inhibition of integrated gastric acidity for all three test products were greater than Prilosec®. The superior control of intragastric pH was particularly evident in the midnight to 3 am period i.e. the period of maximum NAB.

2. The 10/30/10% formulation had much higher plasma concentrations than the other three products from 10 pm to 12 am and slightly higher concentrations from 12 to 1 am. The pharmacodynamic response to these higher concentrations resulted in greater gastric acid suppression from 12-3 am than Prilosec® or the other test formulations.

3. The 10/30/15% plasma concentration and inhibition of integrated gastric acidity was greater than the other three products based on mean plasma profile and the mean point estimates except for 10/30/10% from 12-3 am.

Based on the pharmacokinetic profiles and parameters, the three test formulations performed as designed with a decrease in the pharmacokinetic lag time, a time to peak between 8 pm and 2 am, and an extension of drug release throughout the morning. Additionally, all three two-component formulations had good bioavailability.

These improved pharmacokinetic properties were matched by an improved control of intragastric pH in the peak NAB period of midnight to 3 am.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of controlling nocturnal acid breakthrough in a patient undergoing proton pump inhibitor therapy, the method comprising:
identifying a patient undergoing proton pump inhibitor therapy and exhibiting symptoms of nocturnal acid breakthrough; and
switching said patient from his or her current proton pump inhibitor therapy to a proton pump inhibitor therapy that comprises ingesting, once daily, in the evening, an extended-release proton pump inhibitor formulation comprising a core comprising at least one proton pump inhibitor, which is coated with a pH-dependent coating, which is further coated with a pH-dependent extended release coating,
wherein ingesting the extended-release proton pump inhibitor formulation results in a maximum plasma concentration of the proton pump inhibitor at least two hours after administration.

2. The method according to claim 1, wherein the current proton pump inhibitor therapy comprises ingesting an enterically coated proton pump inhbitor formulation, at least once daily.

3. The method according to claim 2, wherein the current proton pump inhibitor therapy comprises ingesting an enterically coated proton pump inhbitor formulation, at least twice daily.

4. The method according to claim 1, wherein the pH-dependent coating comprises at least one polymer that begins to dissolve at a pH of from about 5 to about 6.

5. The method according to claim 1, wherein the pH-dependent extended release coating comprises at least one polymer that begins to dissolve at a pH of from about 6 to about 7.5.

6. The method according to claim 4, wherein the pH-dependent extended release coating comprises at least one polymer that begins to dissolve at a pH of from about 6.5 to about 7.2.

7. The method according to claim 1, wherein the proton pump inhibitor is omeprazole, an isomer of omeprazole, or a salt of either of the foregoing.

8. The method according to claim 1, comprising switching said patient from his or her current proton pump inhibitor therapy to a proton pump inhibitor therapy that comprises ingesting, once daily, within one hour of an evening meal, the extended-release proton pump inhibitor formulation.

9. A method of controlling stomach acid secretion in a mammal comprising orally administering a pharmaceutical formulation to the mammal, wherein said pharmaceutical formulation comprises at least one proton pump inhibitor structured and arranged to provide a pH-dependent extended-release of a proton pump inhibitor.

10. The method according to claim 9, wherein the pharmaceutical formulation further comprises at least one proton pump inhibitor structured and arranged to provide an initial delayed release of a proton pump inhibitor.

11. The method according to claim 10, wherein the initial delayed release is provided by the presence of a polymer exhibiting a dissolution profile that is pH-dependent.

12. The method according to claim 10, wherein the initial delayed release is provided by a first component and the extended release is provided by a second component.

13. The method according to claim 12, wherein
the first component comprises:
a core comprising at least one proton pump inhibitor, and
a pH-dependent coating;
the second component comprises:
a core comprising at least one proton pump inhibitor,
a pH-dependent coating, and
a pH-dependent extended release coating.

14. The method according to claim 13, wherein the pH-dependent extended release coating comprises at least one polymer.

15. The method according to claim 14, wherein the polymer exhibits a pH-dependent dissolution profile.

16. The method according to claim 15, wherein the polymer exhibits a solubility that is higher at pH 7.25 than at pH 6.8.

17. The method according to claim 16, wherein the polymer exhibits a solubility that is higher at pH 7.25 than at pH 7.15

18. The method according to claim 14, wherein the pH-dependent extended release coating comprises talc.

19. The method according to claim 9, wherein the proton pump inhibitor is omeprazole, an isomer of omeprazole, or a salt of either of the foregoing.

20. The method according to claim 19, wherein the formulation comprises from about 10 to about 60 mg omeprazole, an isomer of omeprazole, or a salt of either of the foregoing, and orally administering the formulation produces a median maximum plasma concentration of omeprazole at greater than about two hours after administration.

21. The method according to claim 20, wherein orally administering the formulation produces a median maximum plasma concentration of omeprazole or an isomer thereof at greater than about two hours to less than about twelve hours after administration.

22. The method according to claim 20, wherein orally administering the formulation produces a median maximum plasma concentration of omeprazole or an isomer thereof at greater than or equal to about four hours after administration.

23. The method according to claim 22, wherein orally administering the formulation produces a median maximum plasma concentration of omeprazole or an isomer thereof at greater than or equal to about four hours to less than about eight hours after administration.

24. The method according to claim 9, wherein the formulation is administered in the evening.

25. The method according to claim 24, wherein the at least one proton pump inhibitor is administered within sixty minutes of an evening meal.

26. The method according to claim 25, wherein the at least one proton pump inhibitor is administered within sixty minutes before an evening meal.

27. The method according to claim 25, wherein the at least one proton pump inhibitor is administered within thirty minutes after an evening meal.

28. The method according to claim 9, wherein the mammal is a human.

29. The method according to claim 28, wherein the proton pump is administered for treatment of gastroesophageal reflux disease.

30. A pharmaceutical formulation for treatment of nocturnal acid breakthrough, the formulation comprising:
an extended component comprising from about 10 to about 60 mg omeprazole, an isomer of omeprazole, or a salt of either of the foregoing, in a core,
the core coated with a coating composition comprising at least one polymer that exhibits a pH-dependent dissolution profile, wherein the polymer exhibits a dissolution that begins at a pH of greater than about 5, to form a coated core, and
the coated core being further coated with an outer coating composition comprising at least one polymer that exhibits a pH-dependent dissolution profile, wherein the polymer exhibits a dissolution that begins at a pH of greater than about 6.5, the outer coating composition further comprising talc.

31. The pharmaceutical formulation according to claim 30, further comprising:
a delayed release component comprising from about 10 to about 20 mg omeprazole, an isomer of omeprazole, or a salt of either of the foregoing, in a core,
said core coated with a coating composition comprising at least one polymer that exhibits a pH-dependent dissolution profile, wherein the polymer exhibits a dissolution that begins at a pH of greater than about 5, to form a coated core.

32. The pharmaceutical formulation according to claim 31, wherein the extended release component comprises 30 mg omeprazole, an isomer of omeprazole, or a salt of either of the foregoing, and the delayed release component comprises 10 mg omeprazole, an isomer of omeprazole, or a salt of either of the foregoing.

33. The pharmaceutical formulation according to claim 31, wherein the extended release component comprises 20 mg omeprazole, an isomer of omeprazole, or a salt of either of the foregoing, and the delayed release component comprises 20 mg omeprazole, an isomer of omeprazole, or a salt of either of the foregoing.
